(19) United States
(12) Patent Application Publication
(10) Pub. No.: US 2006/0100706 A1
(43) Pub. Date: May 11, 2006

(54) Stent systems and methods for spine treatment

(76) Inventors: John H. Shadduck, Tiburon, CA (US); Csaba Truckai, Saratoga, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/130,843

(22) Filed: May 16, 2005

Related U.S. Application Data
(60) Provisional application No. 60/638,970, filed on Dec. 21, 2004.
(60) Provisional application No. 60/640,137, filed on Dec. 29, 2004.
(60) Provisional application No. 60/648,023, filed on Jan. 28, 2005.
(60) Provisional application No. 60/626,701, filed on Nov. 10, 2004.

(51) Int. Cl. A61F 2/44

(52) U.S. Cl. 623/17.11

(57) ABSTRACT

Stent systems and methods for expanding and deploying stents in hard tissue such as bone, more particularly within a vertebral body. One exemplary method includes using a stent body that is coupled to a high speed rotational motor with the stent expandable and detachable from an introducer working end. In one embodiment, the stent is a deformable metal body with zig-zag type struts in an expanded configuration that carries diamond cutting particles bonded to the strut surfaces. The “spin” stent is rotated at high rpm's to remove cancellous bone from the deployment site together with irrigation and aspiration at the end of the probe that carries the stent. The stent may be expanded asymmetrically, such as with first and second balloons or by using an interior restraint, to apply vertical distraction forces to move apart the cortical endplates and support the vertebra in the distracted condition. The cancellous bone about the expanded stent as well as the interior of the stent can be filled with a bone cement, allograft or other bone graft material. In one method of use, the spin stent is designed and adapted for (i) treating a vertebral compression fracture (VCF) or for (ii) reinforcing an osteoporotic vertebral body.

### CROSS-REFERENCE TO RELATED APPLICATIONS

This application claims priority to U.S. Provisional Application No. 60/638,970, filed Dec. 21, 2004, U.S. Provisional Application No. 60/640,137, filed Dec. 29, 2004, and U.S. Provisional Application No. 60/648,023, filed Jan. 28, 2005, the entire contents of which are hereby incorporated by reference in their entirety and should be considered a part of this specification. This application also incorporates by reference U.S. Provisional Application No. 60/626,701 filed Nov. 10, 2004, the contents of which are hereby incorporated herein in its entirety and should be considered a part of this specification.

### BACKGROUND OF THE INVENTION

1. Field of the Invention

Embodiments of the present invention relate to systems and methods for treating hard tissues such as bones, and more particularly, to stent systems for treating fractured or osteoporotic vertebrae that provide for high speed rotational cutting of bone and implantation of an expandable stent in a vertebra to support the vertebra.

2. Description of the Related Art

Osteoporotic fractures are prevalent in the elderly, with an annual estimate of 1.5 million fractures in the United States alone. These include 750,000 vertebral compression fractures (VCFs) and 250,000 hip fractures. The annual cost of osteoporotic fractures in the United States has been estimated at $13.8 billion. The prevalence of VCF in women age 50 and older has been estimated at 26%. The prevalence increases with age, reaching 40% among 80-year-old women. Medical advances aimed at slowing or arresting bone loss from aging have not provided solutions to this problem. Further, the affected population will grow steadily as life expectancy increases. Osteoporosis affects the entire skeleton but most commonly causes fractures in the spine and hip. Spinal or vertebral fractures also have serious consequences, with patients suffering from loss of height, deformity and persistent pain which can significantly impair mobility and quality of life. Fracture pain usually lasts 4 to 6 weeks, with intense pain at the fracture site. Chronic pain often occurs when one level is greatly collapsed or multiple levels are collapsed.

Postmenopausal women are predisposed to fractures, such as in the vertebrae, due to a decrease in bone mineral density that accompanies postmenopausal osteoporosis. Osteoporosis is a pathologic state that literally means “porous bones”. Skeletal bones are made up of a thick cortical shell and a strong inner meshwork, or cancellous bone, of collagen, calcium salts and other minerals. Cancellous bone is similar to a honeycomb, with blood vessels and bone marrow in the spaces. Osteoporosis describes a condition of decreased bone mass that leads to fragile bones which are at an increased risk for fractures. In an osteoporosis bone, the sponge-like cancellous bone has pores or voids that increase in dimension, making the bone very fragile. In young, healthy bone tissue, bone breakdown occurs continually as the result of osteoclast activity, but the breakdown is balanced by new bone formation by osteoblasts. In an elderly patient, bone resorption can surpass bone formation thus resulting in deterioration of bone density. Osteoporosis occurs largely without symptoms until a fracture occurs.

Vertebroplasty and kyphoplasty are recently developed techniques for treating vertebral compression fractures. Percutaneous vertebroplasty was first reported by a French group in 1987 for the treatment of painful hemangiomas. In the 1990's, percutaneous vertebroplasty was extended to indications including osteoporotic vertebral compression fractures, traumatic compression fractures, and painful vertebral metastasis. In one percutaneous vertebroplasty technique, bone cement such as PMMA (polymethylmethacrylate) is percutaneously injected into a fractured vertebral body via a trocar and cannulae system bone biopsy needle. The targeted vertebrae are identified under fluoroscopy. A needle is introduced into the vertebral body under fluoroscopic control, to allow direct visualization. A transpedicular (through the pedicle of the vertebrae) approach is typically bilaterally but can be done unilaterally. The bilateral transpedicular approach is typically used because inadequate PMMA infill is achieved with a unilateral approach.

In a bilateral approach, approximately 1 to 4 ml of PMMA is used on each side of the vertebra. Since the PMMA needs to be forced into the cancellous bone, the technique requires high pressures and fairly low viscosity cement. Since the cortical bone of the targeted vertebra may have a recent fracture, there is the potential of PMMA leakage The PMMA cement contains radiopaque materials so that when injected under live fluoroscopy, cement localization and leakage can be observed. The visualization of PMMA injection and extravasion are critical to the technique—and the physician terminates PMMA injection when leakage is evident. The cement is injected using small syringes to allow the physician manual control of injection pressure.

Kyphoplasty is a modification of percutaneous vertebroplasty. Kyphoplasty involves a preliminary step in the percutaneous placement of an inflatable balloon tamp in the vertebral body. Inflation of the balloon creates a cavity in the bone prior to cement injection. Further, the proponents of percutaneous kyphoplasty have suggested that high pressure balloon-tamp inflation can at least partially restore vertebral body height. In kyphoplasty, the PMMA can be injected at a lower pressure into the collapsed vertebra since a cavity exists, when compared to conventional vertebroplasty.

The principal indications for any form of vertebroplasty are osteoporotic vertebral collapse with debilitating pain. Radiography and computed tomography must be performed in the days preceding treatment to determine the extent of vertebral collapse, the presence of epidural or foraminal stenosis caused by bone fragment retropulsion, the presence of cortical destruction or fracture and the visibility and degree of involvement of the pedicles. Leakage of PMMA during vertebroplasty can result in very serious complications including compression of adjacent structures that necessitate emergency decompressive surgery.

Leakage or extravasion of PMMA is a critical issue and can be divided into paravertebral leakage, venous infiltration, epidural leakage and intradiscal leakage. The exothermic reaction of PMMA carries potential catastrophic consequences if thermal damage were to extend to the dural sac, cord, and nerve roots. Surgical evacuation of leaked cement in the spinal canal has been reported. It has been found that leakage of PMMA is related to various clinical factors such as the vertebral compression pattern, and the extent of the cortical fracture, bone mineral density, the interval from injury to operation, the amount of PMMA injected and the location of the injector tip. In one recent study, close to 50% of vertebroplasty cases resulted in leakage of PMMA from the vertebral bodies. See Hyun-Woo Do et al, “The Analysis of Polymethylmethacrylate Leakage after Vertebroplasty for Vertebral Body Compression Fractures”, Jour. of Korean Neurosurg. Soc. Vol. 35, No. 5 (5/2004) pp. 478-82, (http://wwwjkns.or.kr/htm/abstract.asp?no=0042004086).

Another recent study was directed to the incidence of new VCFs adjacent to the vertebral bodies that were initially treated. Vertebroplasty patients often return with new pain caused by a new vertebral body fracture. Leakage of cement into an adjacent disc space during vertebroplasty increases the risk of a new fracture of adjacent vertebral bodies. See Am. J. Neuroradiol. 2004 February; 25(2):175-80. The study found that 58% of vertebral bodies adjacent to a disc with cement leakage fractured during the follow-up period compared with 12% of vertebral bodies adjacent to a disc without cement leakage.

Another life-threatening complication of vertebroplasty is pulmonary embolism. See Bernhard, J. et al., “Asymptomatic diffuse pulmonary embolism caused by acrylic cement: an unusual complication of percutaneous vertebroplasty”, Ann. Rheum. Dis. 2003; 62:85-86. The vapors from PMMA preparation and injection are also cause for concern. See Kirby, B., et al., “Acute bronchospasm due to exposure to polymethylmethacrylate vapors during percutaneous vertebroplasty”, Am. J. Roentgenol. 2003; 180:543-544.

Another disadvantage of PMMA is its inability to undergo remodeling—and its inability to use the polymer to deliver osteoinductive agents, growth factors, chemotherapeutic agents and the like. Yet another disadvantage of PMMA is the need to add radiopaque agents which lower its viscosity with unclear consequences on its long-term endurance.

In both higher pressure cement injection (vertebroplasty) and balloon-tamped cementing procedures (kyphoplasty), the methods do not provide for well controlled augmentation of vertebral body height. The direct injection of bone cement simply follows the path of least resistance within the fractured bone. The expansion of a balloon applies also compacting forces along lines of least resistance in the collapsed cancellous bone. Thus, the reduction of a vertebral compression fracture is not optimized or controlled in high pressure balloons as forces of balloon expansion occur in multiple directions.

In a kyphoplasty procedure, the physician often uses very high pressures (e.g., up to 200 or 300 psi) to inflate the balloon which first crushes and compacts cancellous bone. Expansion of the balloon under high pressures close to cortical bone can fracture the cortical bone, or cause regional damage to the cortical bone that can result in cortical bone necrosis. Such cortical bone damage is highly undesirable and results in weakened cortical endplates.

In both percutaneous vertebroplasty and kyphoplasty, the injection of polymethylmethacrylate does not create a healthy bone that can respond to normal repetitive stresses. PMMA is simply an inert polymeric monolith that can become brittle when subjected to repeat stresses. A vertebral body thus treated is simply a cortical bone shell that surrounds the hardened polymer infill material.

In both percutaneous vertebroplasty and kyphoplasty, the injection of polymethylmethacrylate further causes osteonecrosis around the PMMA due to the exothermic reaction. The osteonecrosis results in a fibrous capsule around the infill material. Thus, osteonecrosis prevents intercalation of a bone infill material within existing cancellous bone.

Kyphoplasty also does not provide a distraction mechanism capable of 100% vertebral height restoration. Further, the kyphoplasty balloons under very high pressure typically apply forces to vertebral endplates within a central region of the cortical bone that may be weak, rather than distributing forces over the endplate.

There is a general need to provide systems and methods for use in treatment of vertebral compression fractures that provide a greater degree of control over introduction of bone support material, and that provide better outcomes. Embodiments of the present invention meet one or more of the above needs, or other needs, and provide several other advantages in a novel and non-obvious manner.

### SUMMARY OF THE INVENTION

Preferred embodiments of the invention provide stent systems and methods for expanding and deploying stents in hard tissue such as bone. In certain embodiments, the stent systems and methods apply asymmetric distraction forces in bone. In one embodiment, the stent system is designed and adapted for (i) treating a vertebral compression fracture (VCF) or for (ii) reinforcing an osteoporotic vertebral body. One exemplary method includes using a stent body that is coupled to a high speed rotational motor with the stent expandable and detachable from a probe or introducer working end. This “spin” stent may have cutting particles bonded to strut surfaces, and may be rotated at high rpm's to remove cancellous bone from the deployment site together with irrigation and aspiration at the end of the probe.

In one embodiment, the stent is a deformable metal body with zig-zag type struts in an expanded configuration that carries diamond cutting particles bonded to the strut surfaces. The “spin” stent is rotated at high rpm's to remove cancellous bone from the deployment site together with irrigation and aspiration at the end of the probe that carries the stent. Thereafter, the expanded spin stent is de-coupled from the introducer to support the vertebra. Thereafter, the cancellous bone about the expanded stent as well as the interior of the stent can be filled with a bone cement, allograft or other bone graft material for additional support and stabilization of the bone.

In another embodiment, a similar cutting method is used to remove cancellous bone and to deploy the stent. A bone cement is then injected to preserve cancellous bone except that a balloon is expanded to maintain a cavity within the center of the stent and cement. Thereafter, a volume of infill material is injected into the cavity under very high pressures which will distribute forces about cavity, fracture the cement to jack apart endplates to restore vertebral body height while preventing cement flows in unwanted directions.

In another embodiment, the spin stent is fabricated with interwoven struts of wires or ribbons in the form of a Chinese finger-toy with diamond abrasives about the surface of the spin stent for cutting cancellous bone. In all other respects, the methods of this embodiment of the invention are the same with the wire or ribbon forms functioning as the struts of the deformable stent.

In another embodiment, the stent is a deformable metal body with zig-zag type struts that is expanded by first and second elongated balloons during high speed rotation wherein cutting of cancellous bone constrains the stent to a round cross-section. Thereafter, rotation is stopped and the first and second balloons are expanded to provide an asymmetric cross-section for applying vertical distraction forces to move apart the cortical endplates and support the vertebra in the distracted condition. Thereafter, the cancellous bone about the expanded stent as well as the interior of the stent can be filled with a bone cement, allograft or other bone graft material.

In a further embodiment, a stent is expanded by a balloon during high speed rotation wherein cutting of cancellous bone constrains the stent to a round cross-section. Thereafter, rotation is stopped and the balloon is expanded further. The stent body includes interior restraints that extend side-to-side to provide the stent with an asymmetric cross-section when fully expanded to apply vertical distraction forces to move apart the cortical endplates and support the vertebra in the distracted condition. Thereafter, the cancellous bone about the expanded stent as well as the interior of the stent can be filled with a bone cement, allograft or other bone graft material.

Advantageously, preferred embodiments as discussed below provide stent systems that can be deployed in hard tissue rather that in a body lumen. These systems can support and strengthen a damaged vertebrae to carry physiologic loads. The stent when deployed can distribute forces over the cancellous bone to prevent it from being crushed and damaged. More preferably, the stent systems remove cancellous bone to allow the stent to engage cortical bone after partial expansion rather that crushing cancellous bone.

Preferred embodiments also allow for introduction of resorbable polymers for delivering osteoinductive agents, growth factors and chemotherapeutic agents for enhancing bone in-growth.

In certain embodiments, the stent advantageously applies asymmetric forces to bone for augmenting vertebral body height while preventing the application of horizontal forces.

In one embodiment, a method for treating a vertebral body is provided. A stent is inserted into the vertebral body in substantial contact with cancellous bone. The stent is rotated to cut cancellous bone from the vertebral body. The stent is also expanded to support the vertebral body.

In certain preferred embodiments, the stent may be rotated and expanded simultaneously, and can be expanded to a symmetric configuration, an asymmetric configuration, or both in sequence. The stent may be introduced minimally invasively, preferably through a pedicle to the vertebral body. Irrigation and suctioning of the vertebral body may be utilized to remove cut bone debris from the vertebral body. The stent may be expanded into substantial contact with cortical bone endplates of the vertebral body. The method for treating a vertebral body may further comprise inserting the stent with an introducer, and detaching the stent from the introducer following expansion. The stent may be rotated at a speed of between about 100 rpm to about 50,000 rpm. The method may also comprise introducing a fill material through the stent and into the vertebral body. The stent may be expanded using at least one balloon disposed within the stent.

In another embodiment, a plurality of stents may be inserted into the vertebral body in substantial contact with cancellous bone. The stents may be rotated to cut cancellous bone from a treatment site. The stents may be expanded to support the vertebral body.

In another embodiment, a method for treating a vertebral body comprises inserting a stent into the vertebral body. The stent has a collapsed configuration and an expanded configuration. Cancellous bone is cut with the stent. The stent is expanded within the vertebral body. The stent is released such that the stent remains in place to support the vertebral body.

In certain preferred embodiments, the stent may be rotated to cut the cancellous bone. The stent may be expanded simultaneously with cutting the cancellous bone. The stent may be made of metal. The method may further comprise injecting bone cement into the stent after expansion, for example, by directing bone cement through openings in the stent and outside the stent to support the vertebral body. The stent may be inserted on the end of an introducer, and for example, be carried on an inner shaft extending through an elongated shaft of the introducer. The stent may be expanded by drawing proximal and distal ends of the stent closer together. The inner shaft may be rotatable to cut the cancellous bone with the stent. The stent may be released by releasing the introducer from the stent. The method may also comprise delivering a balloon into the expanded stent after injecting bone cement into the stent, and expanding the balloon against hardened bone cement.

In another embodiment, a method for treating a vertebral body comprises inserting a stent into the vertebral body. The stent is expanded asymmetrically such that the stent applies a greater expansion force along an axis extending generally between two cortical end plates of the vertebral body than within a plane generally parallel to the two cortical end plates. The stent is released such that the stent remains in place to support the vertebral body.

In certain preferred embodiments, the stent may be used to cut cancellous bone from within the vertebral body, for example, by rotation of the stent. At least one of irrigation and aspiration may also be used to remove cut bone material. The stent may be expanded symmetrically before expanding the stent asymmetrically. The stent may be expanded with at least one balloon, preferably two balloons. In one embodiment a restraint is provided around at least a portion of the balloon to cause the stent to expand asymmetrically. The stent may be rotated to align the stent before asymmetrical expansion.

In another embodiment, a method for treating a bone is provided. An expandable stent having surface abrasives is introduced into an interior of the bone. The stent is spun to cut the bone, and the stent is expanded. The stent after spinning provides bone support to prevent subsidence.

In certain preferred embodiments, the spinning and expanding the stent occur simultaneously. The method may also comprise irrigating and aspirating cut bone debris. Expansion of the stent may be accomplished by forces applied by at least one of a mechanical stent-expansion mechanism, a balloon stent-expansion mechanism, the release of energy stored in a shape memory stent body, and centrifugal force. The interior of the bone may be filled with at least one of a bone cement, bone allograft or bone autograft. In one embodiment, the bone being treated is a vertebral body.

In another embodiment, a stent is provided comprising a stent body having an outer surface, the body moveable from an unexpanded configuration to an expanded configuration. At least one surface feature is disposed on the outer surface configured to cut bone during rotation of the stent body.

In certain preferred embodiments, the stent body is moveable from a substantially symmetric unexpanded configuration to a substantially symmetric expanded configuration, or to an asymmetric expanded configuration. The body may be moveable first from a substantially symmetric unexpanded configuration to a substantially symmetric expanded configuration, and then from the symmetric expanded configuration to an asymmetric expanded configuration. The body may comprise a slotted wall in the unexpanded configuration, and may comprise a scaffold structure with multiple struts that circumscribe openings in the expanded configuration. In one embodiment, the at least one surface feature is a sharp edge on the struts configured for cutting hard tissue. The at least one surface feature may also be an abrasive material, for example, having a mean dimension ranging form 0.25 micron to 100 microns. The abrasive material may be bonded to the outer surface, and may comprise particles selected from the group consisting of natural monocrystalline diamond, synthetic monocrystalline diamond, polycrystalline diamond and a combination thereof. The stent body may be helically woven, and may be constructed of material having different regions of expandability. The stent may define a longitudinal axis, and the stent when expanded has first and second inwardly convergent ends that converge toward the axis. The stent is preferably sized and configured for insertion into a vertebral body.

In another embodiment, a stent comprises a body having an unexpanded configuration and an expanded configuration. At least one restraining element is coupled to the body, the restraining element configured to substantially constrain the expansion of the body in a first direction and to substantially allow the expansion of the body in a second direction.

In certain preferred embodiments, the restraining element is made of a non-distensible material, and the body comprises a metallic scaffold. The restraining element may include a filament material, a wire material, or a mesh material. The stent may further comprise at least one balloon disposed within the body to expand the stent. The stent may have an outer wall defining openings, and may comprise at least one surface feature disposed on an outer surface of the stent configured to cut bone during rotation of the stent. A plurality of restraining elements may be provided at spaced apart locations along the stent body. The stent is preferably sized and configured for insertion into a vertebral body.

In another embodiment, a stent for treating a vertebral body comprises a scaffolding structure having a proximal end and a distal end and a longitudinal axis defined there between. The structure having an unexpanded configuration and a range of expanded configurations including a first expanded substantially symmetrical configuration about the longitudinal axis and a second expanded asymmetrical configuration about the longitudinal axis.

In certain preferred embodiments, the stent further comprises at least one balloon disposed within the scaffolding structure, the balloon being expandable to move the scaffolding structure from the unexpanded configuration to the expanded configurations. Two balloons may be disposed within the scaffolding structure, the balloons being expandable to move the scaffolding structure from the unexpanded configuration to the expanded asymmetric configuration. A restraint may be provided around at least a portion of the balloon to cause the stent to expand asymmetrically. The scaffolding structure may be made of metal, and may have an outer wall that circumscribes openings to allow bone fill material to pass there through. The scaffolding structure in the second expanded asymmetrical configuration may have a lesser horizontal cross-sectional dimension and a greater vertical cross-sectional dimension. The scaffolding structure in the second expanded asymmetrical configuration may have a generally oblong cross-sectional dimension.

In another embodiment, a stent for treating a vertebral body is provided. The stent comprises a body defining a longitudinal axis extending between a first end and a second end. The stent has an unexpanded configuration to permit its deployment inside a vertebra and an expanded configuration, wherein the body in its expanded configuration increases in size from its first end to an apex portion and then decreases in size to the second end. A plurality of openings extend through an outer surface of the body. At least one cutting feature is disposed on the outer surface of the body. The body is expandable from a substantially symmetrical unexpanded configuration about the longitudinal axis to an asymmetric expanded configuration about the longitudinal axis.

In certain preferred embodiments, the at least one cutting feature is an abrasive material or a sharp edge. The openings may be sized to allow bone fill material to pass there through. In one embodiment, the body is expandable from a substantially symmetrical unexpanded configuration about the longitudinal axis to a substantially symmetrical expanded configuration about the longitudinal axis, and is also expandable from the substantially symmetrical expanded configuration about the longitudinal axis to an asymmetric expanded configuration about the longitudinal axis. A restraining element may be configured to substantially constrain the expansion of the body in a first direction and to substantially allow the expansion of the body in a second direction. The stent may further comprise a balloon for expanding the body.

In another embodiment, a stent system is provided for treating bone. The system comprises a stent movable from an unexpanded configuration to at least one expanded configuration, the stent having surface features configured to cut bone. An introducer is also provided, comprising an elongate body having a proximal end and a distal end, the introducer adapted to releasably engage the stent. A rotation mechanism is configured to rotate the stent, wherein rotation of the stent causes the surface features to engage and cut bone. An expansion mechanism is operatively coupled to the stent and configured to move the stent from the unexpanded configuration to the at least one expanded configuration.

In certain preferred embodiments, the rotation mechanism is adapted to rotate the stent at a speed greater than about 100 rpm, more preferably 500 rpm to about 10,000 rpm. The stent may be symmetric in at least one of the at least one expanded configuration, and may expand radially. The expansion mechanism may be a mechanical expander, such as a screw drive, and may comprise at least one balloon disposed in the stent, the at least one balloon inflatable to move the stent from the unexpanded configuration to the at least one expanded configuration. The surface features may comprise at least one of bone abrading elements and bone cutting elements that may be bonded to a surface of the stent. A fluid source may be coupled to a flow channel in the introducer, the fluid source configured to deliver a fluid to a treatment site adjacent the stent. An aspiration source may be coupled to a flow channel in the introducer, the aspiration source configured to remove debris from a treatment site adjacent the stent. The stent may be made of metal and have a plurality of openings. Fill material may also be provided adapted to be introduced into the expanded stent and through the openings. The introducer is preferably configured to position the stent within a vertebral body.

In another embodiment, a stent system for treating bone is provided. The system comprises a stent moveable from an unexpanded configuration to at least one expanded configuration, and an introducer adapted to be releasably coupled to the stent. A rotation mechanism housed in the introducer is configured to rotate the stent relative to the introducer at a speed of greater than about 100 rpm. An expansion mechanism is operatively coupled to the stent and configured to move the stent from the unexpanded configuration to the at least one expanded configuration.

In certain preferred embodiments, the stent comprises surface features configured to cut bone under rotation. The surface features may be diamond particles bonded to a surface of the stent, or sharp edges on struts of the stent. The stent in the expanded configuration may increase in size from a first end to an apex portion and then decrease in size from the apex portion to a second end. The introducer is preferably configured to position the stent within a vertebral body.

In another embodiment, a system for treating a vertebral body comprises a stent having an unexpanded configuration to permit its deployment inside a vertebra and an expanded configuration. The system also comprises at least one restraining element coupled to the stent, the restraining element configured to substantially constrain the expansion of the stent in a first direction and to substantially allow the expansion of the stent in a second direction. An introducer is also provided, adapted to be releasably coupled to the stent and configured to position the stent within the vertebral body.

In certain preferred embodiments, a rotation mechanism is housed in the introducer and configured to rotate the stent relative to the introducer. An expansion mechanism may also be operatively coupled to the stent and configured to move the stent from the unexpanded configuration to the at least one expanded configuration. The expansion mechanism may include at least one balloon within the stent, the balloon being expandable on opposite sides of the restraining element. The restraining element preferably allows expansion of the body toward cortical ends of the vertebral body.

In another embodiment, a system for treating a vertebral body is provided. The system comprises a stent defining a longitudinal axis, wherein the body is expandable from a substantially symmetrical unexpanded configuration about the longitudinal axis to an asymmetric expanded configuration about the longitudinal axis. An introducer is adapted to be releasably coupled to the stent and configured to position the stent within the vertebral body.

In certain preferred embodiments, the system further comprises a rotation mechanism housed in the introducer and configured to rotate the stent relative to the introducer. An expansion mechanism may also be operatively coupled to the stent and configured to move the stent from the unexpanded configuration to the at least one expanded configuration. The expansion mechanism may comprise at least one balloon disposed in the stent, more preferably two balloons disposed in the stent. The stent may include surface cutting features adapted to cut bone. The stent may be expandable from a substantially symmetrical unexpanded configuration about the longitudinal axis to a substantially symmetrical expanded configuration about the longitudinal axis, and may also be expandable from the substantially symmetrical expanded configuration about the longitudinal axis to an asymmetric expanded configuration about the longitudinal axis. The asymmetric configuration may be provided at least in part by at least one interior restraint coupled to the stent, the interior restraint configured to substantially constrain expansion of the stent in a first direction and substantially permits expansion of the stent in a second direction as the stent is moved into the expanded configuration.

### BRIEF DESCRIPTION OF THE DRAWINGS

In order to better understand embodiments of the invention and to see how they may be carried out in practice, some preferred embodiments are next described, by way of non-limiting examples only, with reference to the accompanying drawings, in which like reference characters denote corresponding features consistently throughout similar embodiments in the attached drawings.

FIG. 1 is a side view of a spine segment with one vertebra having a vertebral compression fracture (VCF).

FIG. 2A is a cross-sectional view of a vertebra with the working ends of two stent deployment systems disposed therein for the removal of cancellous bone and stent deployment, in accordance with one embodiment.

FIG. 2B is a cross-sectional view of a vertebra with the working end of one stent deployment system disposed therein for the removal of cancellous bone and stent deployment, in accordance with another embodiment.

FIG. 3 is a perspective view of a stent deployment system, in accordance with one embodiment, having an expandable stent at one end thereof.

FIG. 4A is an enlarged cut-away view of one embodiment of an expandable stent in a first configuration for introduction into cancellous bone.

FIG. 4B is a cut-away view of the expandable stent of FIG. 4A in a second partly expanded configuration while rotated to remove cancellous bone.

FIG. 4C is a cut-away view of the expandable stent of FIGS. 4A and 4B in another expanded configuration while rotated to remove additional cancellous bone.

FIG. 4D is a cut-away view of the stent of FIGS. 4A-4C in an expanded configuration illustrating the inflow of bone cement through the stent to interdigitate with, and preserve, cancellous bone.

FIG. 4E is a cut-away view of the stent of FIGS. 4A-4D with the stent and/or working end of the stent deployment system de-coupled from the introducer portion of the system.

FIG. 5A is a side view of one embodiment of an expandable stent that retains an open interior cavity after injection of an in-situ hardenable bone cement to infiltrate and preserve cancellous bone.

FIG. 5B is a side view of the expandable stent illustrated in FIG. 5A illustrating the injection of additional infill material under high pressure to fracture the cement, deform the cement and apply vertical distraction forces to endplates to augment the vertebral height.

FIG. 6 is a cut-away view of another embodiment of an expandable stent.

FIG. 7 is a perspective view of another embodiment of a stent deployment system having an expandable stent at one end thereof.

FIG. 8A is a schematic side view of another embodiment of an expandable stent in an expanded configuration.

FIG. 8B is a schematic side view of another embodiment of an expandable stent in an expanded configuration.

FIG. 8C is a schematic side view of another embodiment of an expandable stent in an expanded configuration.

FIG. 9 is a partial cross-sectional view of the handle of one embodiment of a stent deployment system having an asymmetric stent.

FIG. 10A is a schematic view of one embodiment of an asymmetric stent in a collapsed configuration.

FIG. 10B is a schematic view of an asymmetric stent rotated from a collapsed configuration.

FIG. 10C is a schematic view of the asymmetric stent of FIGS. 10A-10B rotated to an expanded configuration.

FIG. 10D is a schematic view of the asymmetric stent of FIGS. 10A-10C in another expanded configuration.

FIG. 11 is a cross-sectional view of one step in a method of deploying an expandable stent and removing cancellous bone from a vertebra, in accordance with one embodiment.

FIG. 12 is a cross-sectional view of another step in a method of deploying an expandable stent and removing cancellous bone from a vertebra, where the stent has expanded to remove cancellous bone to the interface with cortical bone.

FIG. 13 is a cross-sectional view of another step in a method of deploying an expandable stent and removing cancellous bone from a vertebra, where the stent is expanded asymmetrically to elevate vertebral height.

FIG. 14 is a cross-sectional schematic view of another embodiment of a stent deployment system having first and second tapered balloons for creating vertical distraction forces.

FIG. 15 is a view of an asymmetric pattern of one embodiment of an asymmetric expandable stent.

FIG. 16 is an enlarged view of an asymmetric pattern of a wall of the expandable stent illustrated in FIG. 15.

FIG. 17A is a longitudinal cross-sectional schematic view of another embodiment of an expandable stent having an interior restraint structure, the stent in a partially expanded configuration.

FIG. 17B is a transverse cross-sectional view of the stent of FIG. 17A taken along line 17B-17B of FIG. 17A.

FIG. 18A is a longitudinal cross-sectional schematic view of the stent of FIGS. 17A-17B in an asymmetric expanded configuration.

FIG. 18B is a transverse cross-sectional view of the stent of FIG. 18A taken along line 18B-18B of FIG. 18A.

FIG. 19A is a sectional perspective view of one embodiment of an expandable stent in a partially deployed configuration.

FIG. 19B is a sectional perspective view of the stent of FIG. 19A in an expandable configuration.

FIG. 20A is a cross-sectional view of one step in a method of deploying an expandable stent and removing cancellous bone from a vertebra, in accordance with one embodiment.

FIG. 20B is a cross-sectional view of another step in a method of deploying an expandable stent and removing cancellous bone from a vertebra, where the stent has been expanded to cut cancellous bone to the interface with cortical bone.

FIG. 20C is a cross-sectional view of another step in a method of deploying an expandable stent and removing cancellous bone from a vertebra, where the stent is expanded asymmetrically to elevate vertebral height.

### DETAILED DESCRIPTION OF THE PREFERRED EMBODIMENTS

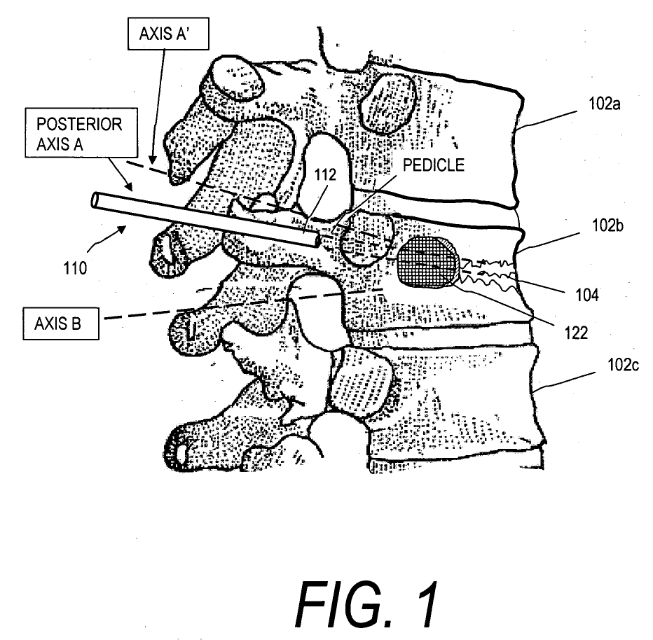

FIG. 1 illustrates a vertebral body 102b with a wedge vertebral compression fracture (VCF) 104. The stent deployment systems and methods disclosed herein are directed in some embodiments to safely introducing bone cement into cancellous bone to eliminate pain and to increase vertebral body height. Vertebral body 102a is susceptible to a VCF following treatment of the fractured vertebra 102b since biomechanical loading will be altered. More particularly, the stent deployment systems and methods disclosed herein include systems for treating an acute or older VCF and for preventing a future VCF in a spine segment. In particular, the systems are adapted for restoration of vertebral body height to thereby restore biomechanics of the affected spine segment.

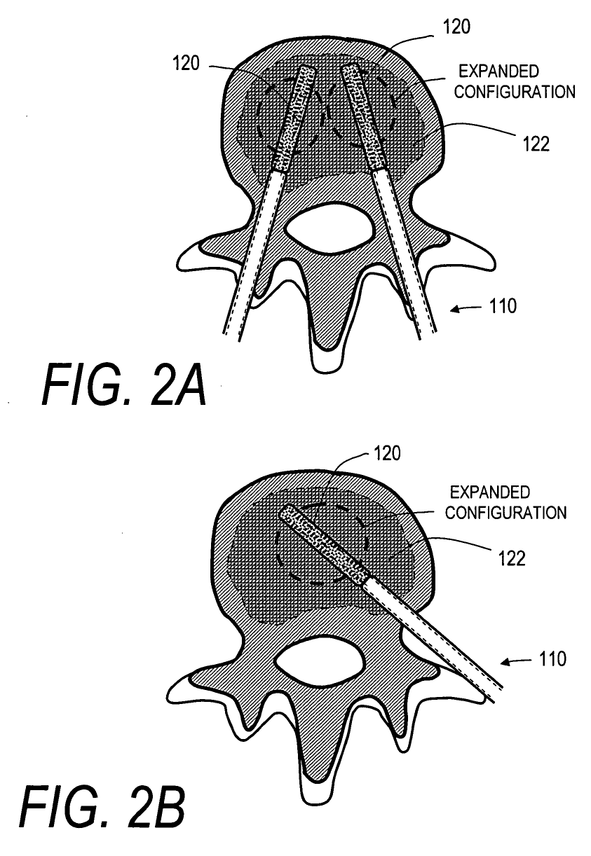

FIGS. 1, 2A and 2B illustrate one embodiment of a stent deployment system 100 comprising a probe or introducer 110 having an elongated shaft 112 that carries an expandable stent 120 (not shown in FIG. 1) at a distal end of the probe 110. In FIG. 2A, two probes 110 are inserted through the saddles of the pedicles of a vertebra along generally posterior axes A and A′ to deploy the expandable stents 120 into the osteoporotic cancellous bone 122 of the vertebral body. As shown in FIG. 2B, the probe 110 can also be introduced at other locations, such as through the wall of the vertebral body along axis B (see FIG. 1), or in an anterior approach (not shown).

In a preferred embodiment, the stent deployment system 100 is introduced into the vertebral body 102a in a minimally invasive manner. For example, the probe or introducer 110 can be introduced in a transpedicular approach as is known in the art. In another embodiment, access to the affected vertebral body 102a can be provided via a regular open surgical procedure known in the art.

A stent is normally considered to be a tubular support member having a cylindrical lumen for holding open a vessel lumen in a human body, or for longitudinally connecting the lumens of portions of vessels adjacent to one another. In this disclosure, the term “stent” is used at times for convenience to describe the apparatus corresponding to one of the embodiments disclosed herein since the apparatus ultimately serves the function of a support member to maintain distraction forces in a bone, such as a vertebra, with abnormalities therein. The stent is also referred to at times as a “rotatable” or “spin” stent, or a “cutting” stent to describe its function as a cutting instrument as well as a support member. Certain embodiments of stents according to the disclosed embodiments differ greatly from prior art stent designs that support body lumens, in that these stents as disclosed herein are rotated or spun at very high speeds to cut, grind or otherwise remove bone or hard tissue to accommodate the expanded stent body. In contrast, other prior art stents are adapted for soft tissue retraction, that may include the fracture of hardened occlusive materials. As used herein, the term “stent” is a broad term that encompasses its ordinary meaning and includes, but is not limited to, stents such as described above and stents as described further below, and any expandable, implantable structure that serves the function of a support member to maintain distraction forces in a bone.

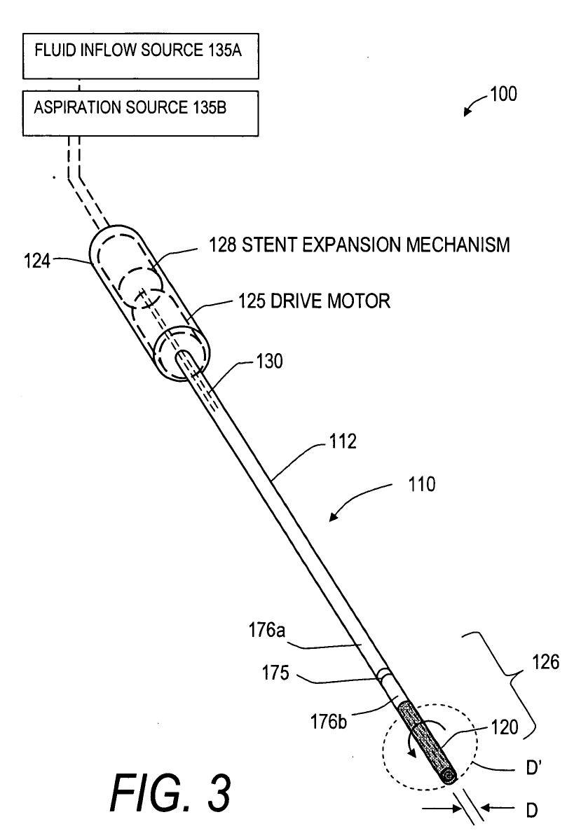

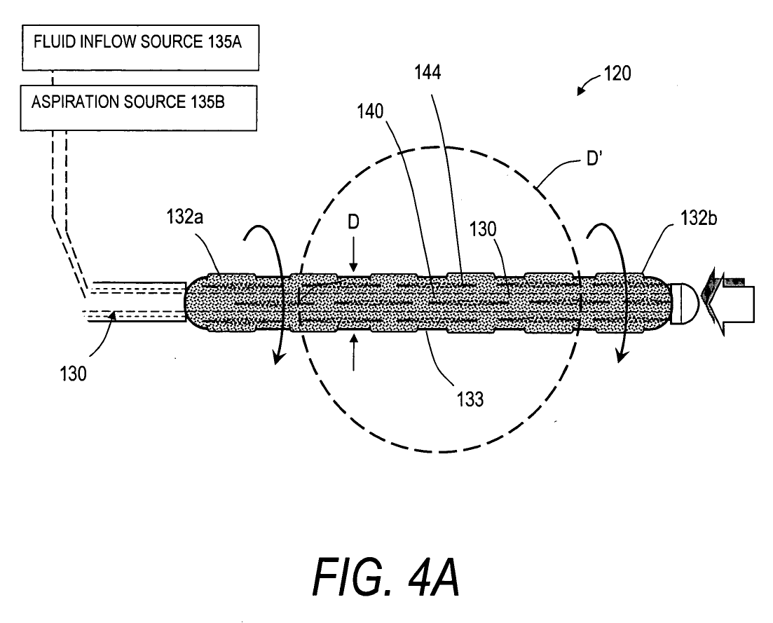

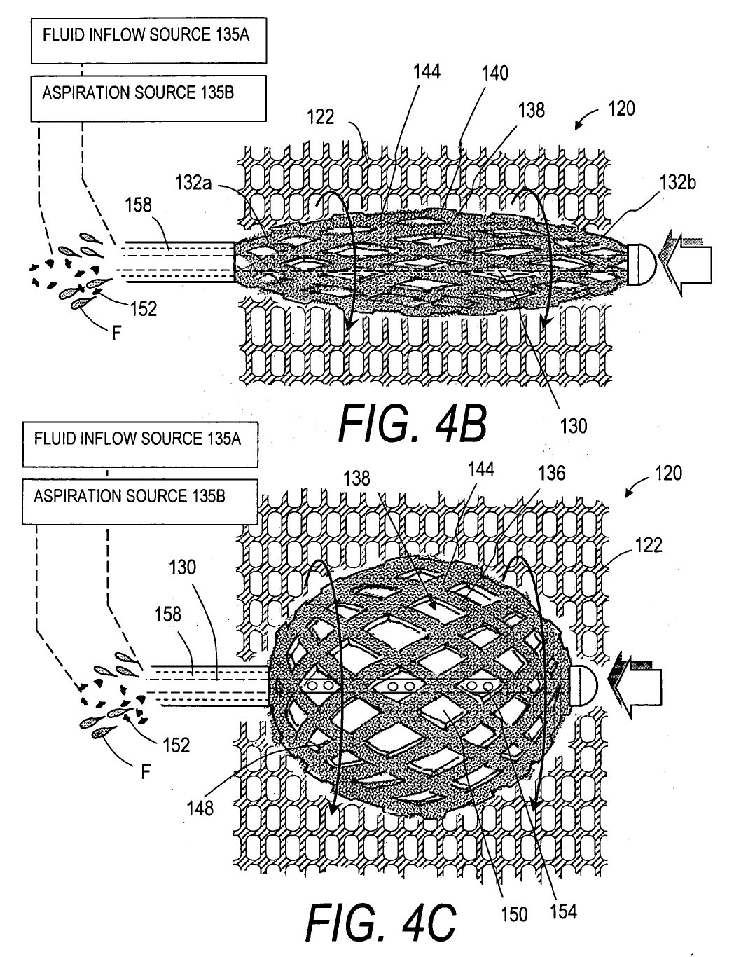

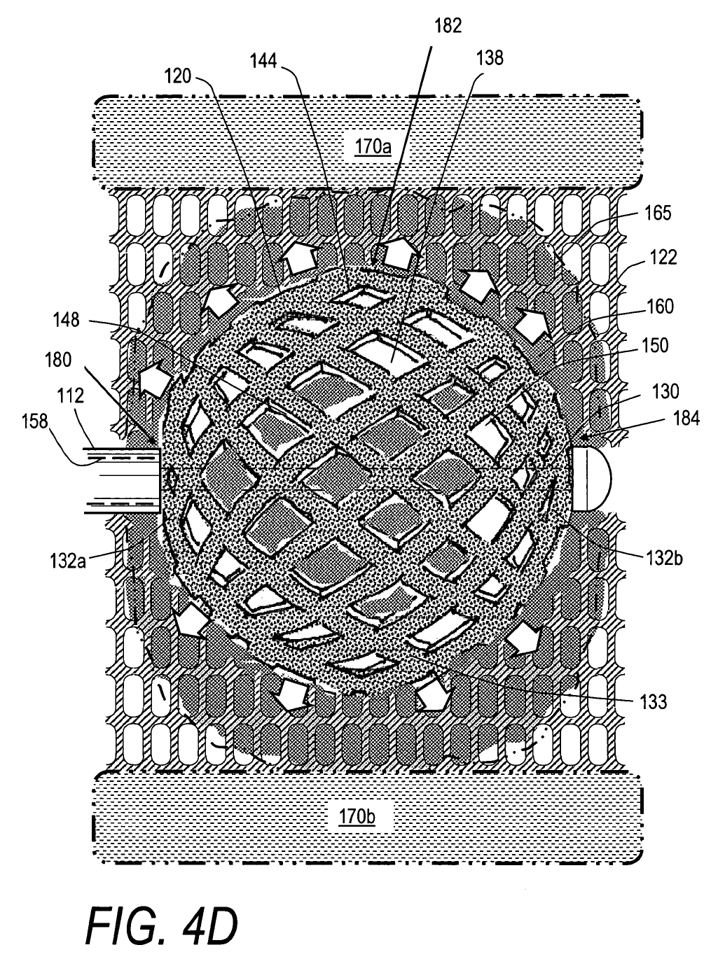

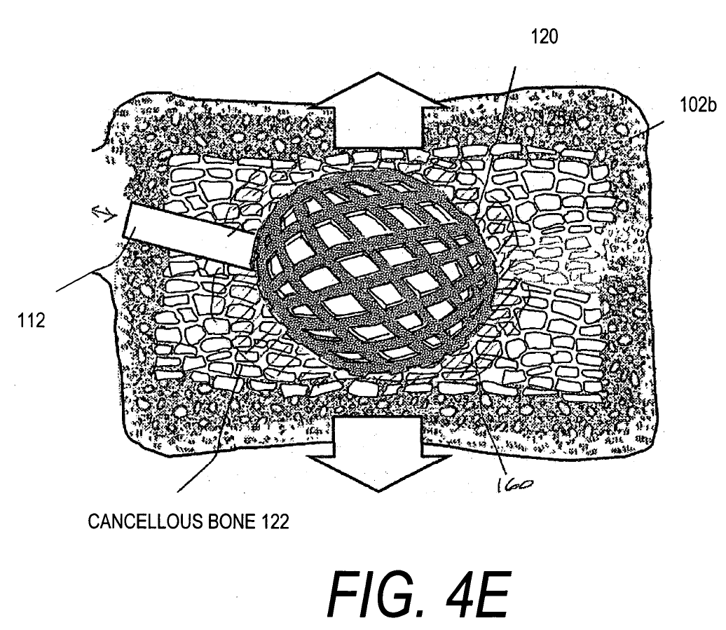

With reference to FIGS. 3 and 4A-4B, the use of the probe 110 and cutting or spin stent 120 is shown. As shown in FIG. 3, the probe 110 has a handle portion 124 that houses a drive motor 125 for rotating the stent 120 carried at a working or distal end 126 of the stent deployment system 100. Any suitable rotation mechanism, such as an electric motor or an air motor, may be used. In a preferred embodiment, the stent 120 is carried on an inner shaft or sleeve 130 that is rotatable at a high speed within the elongated shaft 112. The handle 124 further includes an expansion mechanism 128 for expanding the stent body 120 from a pre-deployed non-expanded condition, shown in FIG. 3, to an expanded condition, as indicated sequentially in FIGS. 4A-4D.

In a preferred embodiment, the stent body 120 has enough support strength to maintain its expanded shape even against compressive forces applied within the bone. For example, the stent in one embodiment may be irreversibly expanded, such that it locks in an expanded configuration once expanded. In another embodiment, the stent may be deployable such that it can be actuated from the expanded configuration back to its collapsed configuration, if desired, for example, to remove or reposition the stent if improperly placed. In one embodiment, additional support strength may be provided to the expanded stent 120 via cement introduced through the stent 120, as further discussed below.

In one exemplary embodiment, the inner sleeve 130 can be a pull-rod 130 and part of the expansion mechanism 128 (see FIGS. 3 and 4A), which is moved axially to compress the ends 132a, 132b of the stent 120. The inner sleeve 130 may be connected to the distal end of the stent, with an enlarged cap provided distal to the stent, and may be pulled proximally, while the proximal end of the stent engages the distal end of the introducer 110 to cause the stent to expand. Said axial compression flexes the central stent region 133 outwardly. In one embodiment, the stent expansion mechanism 128 can be a motorized screw drive that translates pull-rod 130. However, the stent expansion mechanism 128 can be any suitable mechanism for expanding the stent 120. In one embodiment, the drive motor and stent expansion mechanisms 125, 128 are linked for simultaneous operation. In another embodiment, one of the mechanisms 125, 128 can be operated by a handswitch in the probe 110 and the second of the mechanisms can be operated, for example, by a footswitch. One of ordinary skill in the art will recognize that other suitable actuation means can be used.

As illustrated in FIG. 3, the probe handle portion 124 is preferably also operatively coupled to a fluid inflow source 135A and a cooperating aspiration source 135B for the extraction of cut materials from the targeted bone treatment site. In one embodiment, the irrigation/aspiration sources 135A, 135B can also be slaved to the other operational mechanisms 125 and 128 described above. In another embodiment the irrigation/aspiration sources 135A, 135B can be operated manually. As described further below, irrigation fluid can be provided to the treatment site through a lumen in the inner sleeve 130, while the fluid can be aspirated through the annulus between the inner sleeve 130 and the shaft 112.

In one embodiment, as illustrated in FIGS. 4B and 4C, the stent 120 comprises a metal scaffold with struts 136 that circumscribe openings 138. The struts 136 are preferably made of deformable metal and have a zig-zag configuration upon expansion. For example, in one embodiment, the stent 120 can be made of stainless steel, nickel titanium alloy, titanium, tantalum, combinations thereof, or other suitable metal or metal alloy. However, the stent 120 can be made of any suitable material in any suitable configuration for use in the treatment of vertebral bodies.

The stent 120 preferably has a structure that is collapsible to a suitable diameter D, akin to a slotted tube, as in FIG. 3, with a diameter at a transverse cross-section in a range of between about 3 mm and about 5 mm. In an expanded condition, as shown in FIG. 4C, the stent 120 can have a diameter D′ at a transverse cross-section in a range of between about 10 mm and about 20 mm. Preferably, the stent 120 can have any suitable longitudinal planform in the expanded configuration, such as round, elliptical, cylindrical, or polygonal. As illustrated in FIG. 4D, in one embodiment the stent 120 when expanded increases in dimension from a proximal end 180 to an apex portion 182, and then decreases in dimension from the apex portion 182 to a distal end 184. In the embodiment of FIG. 4D, because the stent 120 in longitudinal planform is generally round or elliptical, the apex portion 182 is located at a single transverse plane. However, it will be appreciated that the apex portion 182 may also comprise a cylindrical portion, such that the expanded stent 120 will have a substantially constant cross-sectional area over a length between the proximal and distal ends. In other embodiments, the expanded stent may have a substantially constant cross-sectional area from the proximal end to the distal end.

As shown in FIGS. 3 and 4A, in a preferred embodiment the stent 120 has an outer surface 140 that includes surface features 144 adapted for the abrasive removal, grinding or cutting of cancellous bone upon high speed rotation of the stent 120. In one embodiment, the stent 120 is rotated and expanded simultaneously. In another embodiment, the stent 120 is expanded intermittently while said rotation is stopped. In one embodiment, the surface features 144 comprise abrasive surface features affixed to the surfaces 140, such as abrasive particles of diamond, carbide or other suitable materials. Diamond particles can be natural monocrystalline diamond, synthetic monocrystalline diamond, polycrystalline diamond or a combination thereof. Preferably, diamond particles have a mean dimension ranging from between about 0.25 micron and about 100 microns, and more preferably from between about 1 micron and about 50 microns. However, the surface features 144 can comprise any material suitable for the removal of cancellous bone. In one embodiment, the abrasive surface features 144 can be bonded onto the stent 120, such as via an adhesive. In one preferred embodiment, the struts 136 have edges or apexes 148 that are sharp and function as surface cutting features.

As illustrated in FIG. 3, a first end of the inner sleeve 130 is preferably operatively coupled to the drive motor 125 for rotating the inner sleeve 130 at a speed ranging from between about 100 rpm and about 50,000 rpm or more, and more preferably at a speed ranging from between about 500 rpm and about 10,000 rpm. In another embodiment, spinning the stent 120 at high speeds also applies substantial centrifugal forces, contributing to the expansion of the stent 120.

As illustrated in FIGS. 4B-4D, the stent 120 preferably has an open central portion 150 wherein abrasion debris 152 and fluid F can be aspirated from the treatment site using the aspiration source 135B. In the illustrated embodiment, the fluid inflow source 135A has infusion ports 154 disposed generally along or at a distal end of the inner sleeve 130, through which irrigation fluid can be delivered to the treatment site. Additionally, as shown in FIGS. 4B-4C, the aspiration source 135B can be used to suction debris from the treatment site through the lumen or bore 158 in the shaft 112 of the probe 110. One of ordinary skill in the art will recognize that irrigation fluid need not be delivered through the illustrated infusion ports 154, but can instead be delivered to the treatment site using other mechanisms, such as via the through bore in the shaft 112. Likewise, one of ordinary skill in the art will recognize that debris need not be suctioned through the illustrated through bore 158, but can instead be suctioned from the treatment site via other mechanisms, such as through the suction ports 154 on the inner sleeve 130 extending through the stent 120.

Referring again to FIGS. 4B-4D, an exemplary method for treating a vertebral body is shown. In the illustrated embodiment, the stent 120 has an unexpanded configuration and an expanded configuration. Additionally, the surface cutting features 144 are capable of high speed rotational reduction of cancellous bone.

The rotatable or spin stent 120 is introduced into cancellous bone in the unexpanded configuration. In one preferred embodiment, the rotatable stent 120 is contemporaneously rotated and expanded to reduce cancellous bone. In another embodiment, the stent 120 is intermittently rotated and expanded. The rotation of the stent 120 causes the cutting features 144 on the stent to cut away cancellous bone, which can be removed through the aspiration channel 158. Preferably, the stent 120 is in an expanded configuration to support the bone of the vertebral body when rotation of the stent 120 is ceased. In particular, the deployment of the spin stent 120 preferably supports the bone to prevent its subsidence or tendency to move toward a non-distracted condition. The bone can subsequently be infilled with bone cement or bone graft material, as discussed below.

FIG. 4D shows filler material 160 introduced into the open central portion 150 of the stent 120 following expansion of the stent 120. The filler material preferably flows through the openings 138 in a plume 165 to thereby intercalate with cancellous bone 122. Depending on the selected diameter of stent 120 in its expanded configuration, the stent 120 can cut to the superior and inferior cortical bone (endplate) layers 170a, 170b of the vertebra to provide distraction to cortical bone. In another embodiment, the stent 120 can have a diameter D′ in the expanded configuration that is somewhat smaller than the distance between the cortical bone layers 170a, 170b, leaving a margin of cancellous bone around the stent 120.

Preferably, a hardenable cement 160 is introduced through the openings 138 of the stent 120 to preserve the remaining cancellous bone. In one preferred embodiment, the hardenable cement 160 is PMMA. However, the hardenable cement 160 can comprise other suitable materials configured to preserve cancellous bone, as further discussed below. Preferably, the dimensions of the stent 120 are designed so that the inflow material will flow to reinforce the cancellous bone as it transitions into the cortical layers, or so that the cement will fully engage the cortical bone layers 170a, 170b.

FIG. 4E illustrates the stent 120 decoupled from the probe 110. As shown in FIG. 3, in one embodiment the introducer or probe 110 can optionally have a release or detachment structure 175 for de-mating the working end 126 from a proximal portion 176a of the introducer 110. In one preferred embodiment, the detachment structure 175 also detaches the inner sleeve 130 in addition to the shaft 112 at the working end 126 from the inner sleeve 130 at the proximal portion 176a. Accordingly, a portion of the inner sleeve 130 remains in the stent body 120. Alternatively, the inner sleeve 130 can releasably engage the distal end of the stent, and once the stent is expanded, the inner sleeve 130 can be released from the stent by mechanisms such as disclosed herein. The release or detachment structure 175 can be any suitable mechanism, such as a screw thread, a releasable clamp, a thermally sacrificial polymer, a fracturable element, or a scored frangible structure that is broken by extension forces. In another embodiment, the detachment structure 175 can include the system invented for spacecraft, which can be adapted for medical use. In this embodiment, the system is known in the art as a nickel titanium (NiTi) actuated frangibolt system, which was developed to replace explosive bolts in satellite deployment. The system would include a resistively heated NiTi actuator to separate the implantable medical device working end 126 from the introducer 110 or catheter based on frangibolt designs disclosed in U.S. Pat. No. 5,119,555, the entirety of which is hereby incorporated by reference, and commercialized by TiNi Aerospace, Inc., 2235 Polvorosa Drive, Suite 280, San Leandro, Calif. 94577; see also http://www.tiniaerospace.com/frangibolt.html. The system thus de-couples the proximal portion 176a of the introducer 110 from the distal portion 176b thereof, leaving the working end 126 and the stent 120 in the vertebra, as illustrated in FIG. 4E. In another embodiment, the stent 120 alone can be released from the probe or introducer 110.

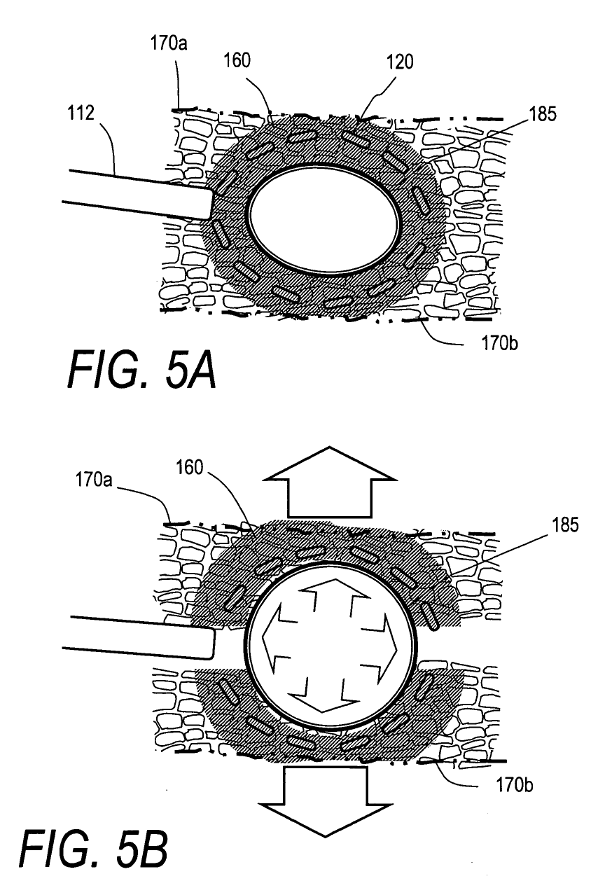

In accordance with another embodiment, the expanded stent 120, as in FIG. 4D, can be infilled with bone cement 160 as shown in FIG. 5A. In the illustrated embodiment, a balloon 185, or other similar structure, is disposed in the stent 120 and opened or expanded, causing the bone cement 160 to form a hardened region about the periphery of the stent 120 and in the cancellous bone to preserve the cancellous bone. The balloon is preferably part of the implanted structure, and may be provided over the inner sleeve 130 which remains within the stent after deployment. Preferably, the balloon 185 is further expanded under a pressure sufficient to break the just-hardened cement 160, as shown in FIG. 5B. In one embodiment, the balloon 185 is made of a material configured to apply very high pressures to break the hardened cement 160, such as PET or urethane. The further expansion of the balloon 185 additionally applies distraction forces to augment the vertical height of the vertebra. In a preferred embodiment, the stent 120 is used to remove cancellous bone so that the stent surfaces engage the cortical endplates 170a, 170b. Further details on balloon-expanded stents are provided below.

In the embodiments described above, the high pressure inflows of infill material can inject any type of bone cement or autograft, allograft or the like. In the illustrated embodiments, the stent 120 can be designed (i) to support physiologic loads on the vertebra, or (ii) to temporarily support loads intraoperatively with the infill material in combination with the stent later functioning to support physiologic loads.

In another embodiment, the stent 120, as shown in FIGS. 4A-4E, can be expanded by a balloon expansion member (not shown) rather than a pull-rod 130. The method of stent deployment using said balloon expansion mechanism would preferably remain the same, as illustrated in FIGS. 4A-4E. However, irrigation and suction using the fluid flow and aspiration sources 135A, 135B would operate over the surface of the stent 120 and balloon structure.

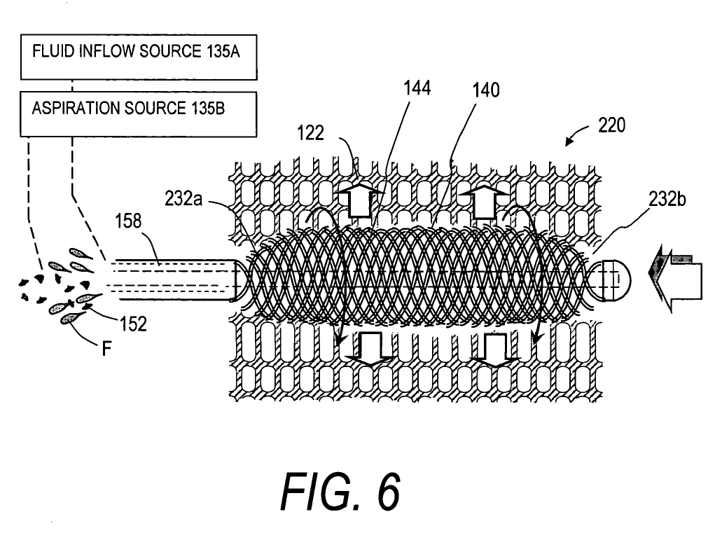

In another embodiment shown in FIG. 6, a rotatable stent can be a stent 220 made of wire-like elements. In the illustrated embodiment, the stent 220 has a helically woven structure. Preferably, such a structure is made of a shape memory material or superelastic material such as nickel titanium (NiTi), and is deformable with a memorized shape in the expanded configuration for supporting bone after deployment and expansion. The movement toward the memorized shape can be assisted or actuated by a central pull-wire or by balloon expansion means, such as the embodiments discussed above. Alternatively, the self-expansion force created by the stent 220 may be sufficient to apply forces to support the bone. Centrifugal forces caused by the high speed rotation of the stent 220 may also be used to support the bone. In a preferred embodiment, each wire-like element can be a cutting wire, for example, with the abrasive particles or other surface features 144 bonded thereto. In all other respects, the stent 220 would be operated as depicted in FIGS. 4A-4E. In one embodiment, the stent 220 can comprise wire or ribbon material such as formed from superelastic NiTi, and for example, in the form of a Chinese finger-toy. In such an embodiment, the stent body can preferably be progressively deployed from a tubular introducer as the stent body is rotated, which can allow the deployment of a more elongated stent body.

The stent 220 has a proximal end 232a and a distal end 232b, with the proximal end 232a releasably attached to the distal end of the shaft 112, and the distal end releasably attached to the distal end of the inner sleeve 130. The stent 220 may be releasable from the sleeves by any of the mechanisms previously described.

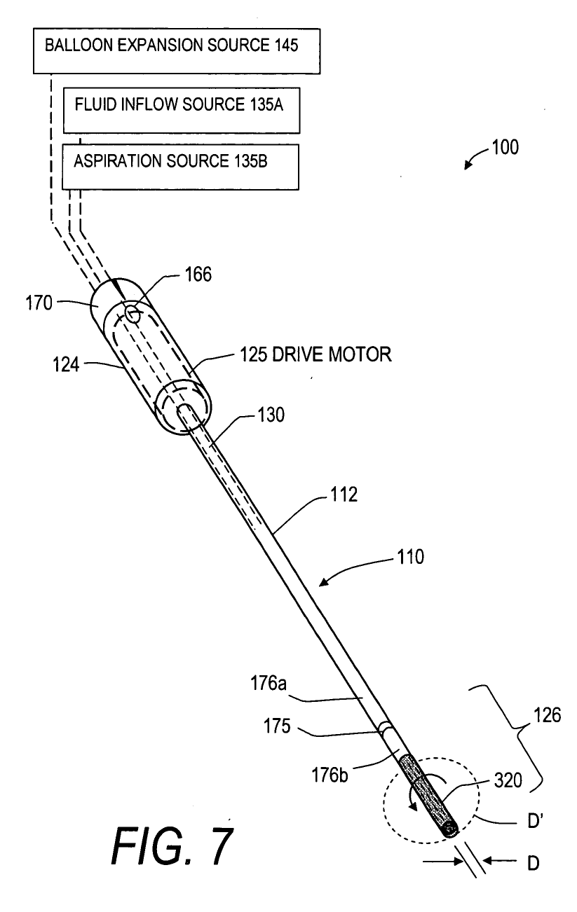

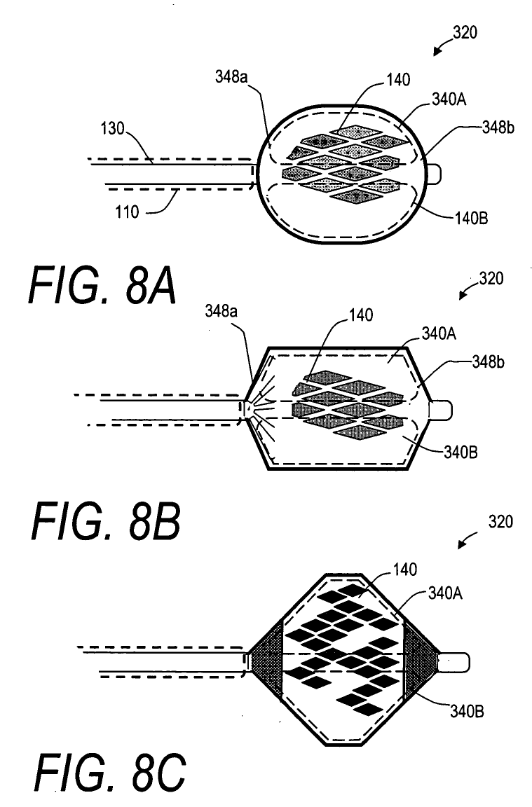

With reference to FIGS. 7 and 8A-8C, another embodiment of a stent deployment system 100 is shown having a rotatable asymmetric stent 320 in a pre-deployed configuration. As described in the preferred embodiments herein, the asymmetric stent is asymmetrical about the longitudinal axis of the stent (i.e., the axis extending between its proximal and distal ends), such that the stent expands more in a first transverse direction relative to the longitudinal axis than in a second transverse axis relative to the longitudinal axis, the second transverse axis being perpendicular to the first transverse axis. In the context of a vertebral body, the stent desirably applies a greater expansion force along an axis extending generally between two cortical end plates of the vertebral body (in other words, vertically) than within a plane generally parallel to the two cortical end plates (in other words, horizontally). In other embodiments the asymmetric stent can be asymmetric about a transverse axis, such that the stent expands more proximally or distally. The embodiment of the introducer or probe 110 illustrated in FIG. 7 is similar to the embodiment illustrated in FIG. 3, and similar components in both embodiments are identified with the same numerical identifier. As used herein, asymmetric refers to the ability of the stent 320 to be expanded into an asymmetric configuration, preferably from a generally symmetric configuration.

As shown in FIG. 7, it can be seen that the stent deployment system 100 includes the introducer or probe 110, a handle 124, a balloon expansion source 145, a fluid inflow source 135A and an aspiration source 135B. The probe 110 includes an elongate shaft 112, and is provided with an elongate inner shaft 130 extending to a distal end thereof. The stent 320 is mounted on the distal end of the shaft 130 as described above. The balloon expansion source is provided for expanding at least two expandable balloons 340A, 340B disposed within the stent body 320, as further described below. The balloons may be mounted to the inner sleeve 130, with the inflation lumens for the balloons provided within the inner sleeve. The expansion source 145 supplies a pressurized fluid to expand the balloons 340A, 340B, and thus the stent body 320, from a pre-deployed or non-expanded configuration, as shown in FIG. 7, to an expanded configuration, as illustrated sequentially in FIGS. 8A-8C. One of ordinary skill in the art will recognize that the stent deployment system 100 can have one balloon or more than two balloons disposed in the stent body 320 and that the pressurized fluid can be a liquid or a gas. In a preferred embodiment, the balloons 340A, 340B are made of a non-distensible material known in the art, such as PET or urethane, or other suitable materials known in the art of medical dilation balloons.

In the embodiments shown in FIGS. 8A-8C, the expanded asymmetric stent 320 preferably has first and second ends 348a, 348b that converge, or taper toward the longitudinal axis of the stent, and couple to the shaft portion or inner sleeve 130. When in the expanded configuration, the balloons 340A, 340B and stent body 320 may have a longitudinal cross-sectional profile that is round, oval, or generally angular. FIG. 8A illustrates a stent that is generally oval or oblong in its longitudinal cross-sectional profile. FIG. 8B illustrates a stent that has a generally constant, cylindrical apex portion over a majority of the length of the stent. FIG. 8C illustrates a stent with a shorter apex portion. However, the balloons 340A, 340B and stent body 320 can have any suitable longitudinal cross-sectional profile when in the expanded configuration, such as polygonal.

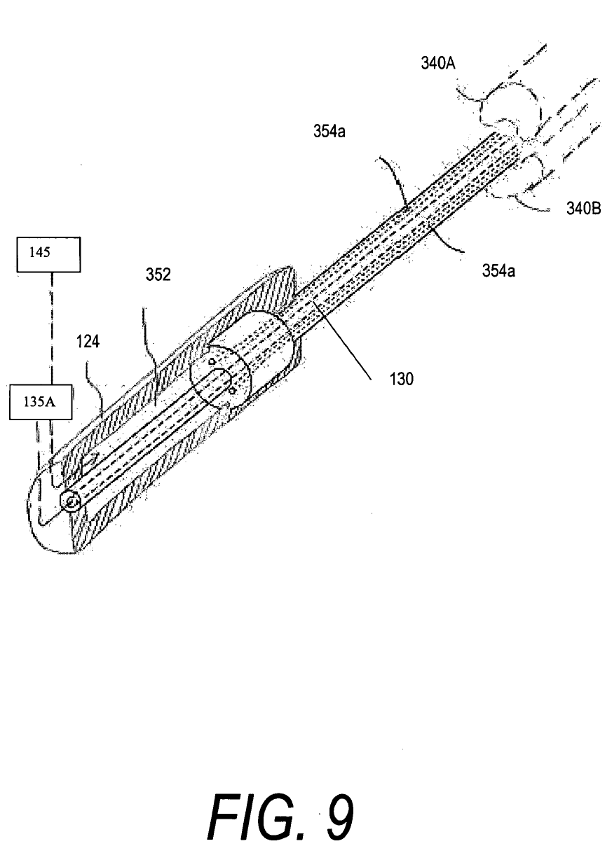

FIG. 9 depicts a cut-away schematic view of the handle 124 with the rotatable shaft or inner sleeve 130 that carries the two expandable balloons 340A, 340B and that rotates the stent 320. In the illustrated embodiment, fluid from the expansion source 145 enters a chamber 352 that communicates via lumens 354a, 354b with the balloons 340A, 340B. In one embodiment, the expansion source 145 provides the fluid through the lumens 354a, 354b to inflate the balloons 340A, 340B as the shaft 130 spins. In another embodiment, the expansion source 145 provides said fluid to inflate the balloons 340A, 340B during intermittent stops in the rotation of the stent 320. In one embodiment, the fluid is a liquid. In another embodiment, the fluid is a gas. The motor drive mechanism 125 and the aspiration source 135B are not shown for convenience in FIG. 9.

As with the embodiment of FIG. 3 above, the probe handle portion 124 used in connection with stent 320 as shown in FIG. 7 is operatively coupled to a fluid inflow source 135A and a cooperating aspiration source 135B for extraction of cut materials from the targeted bone treatment site, for example through a lumen in shaft 112. The irrigation/aspiration system can also be slaved to the rotation motor 125 and the balloon expansion source 145 described above or can be operated manually. In operation, the fluid inflows would be introduced at a first end of the balloons-stent assembly and then extracted at the opposing end of the assembly.

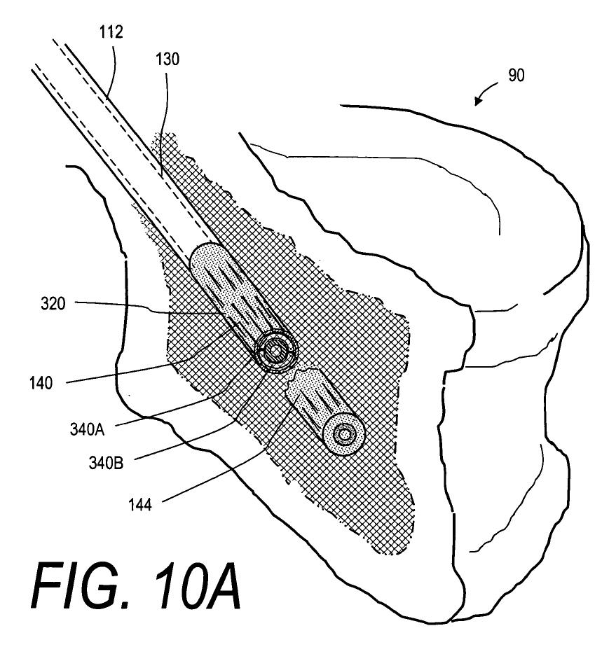

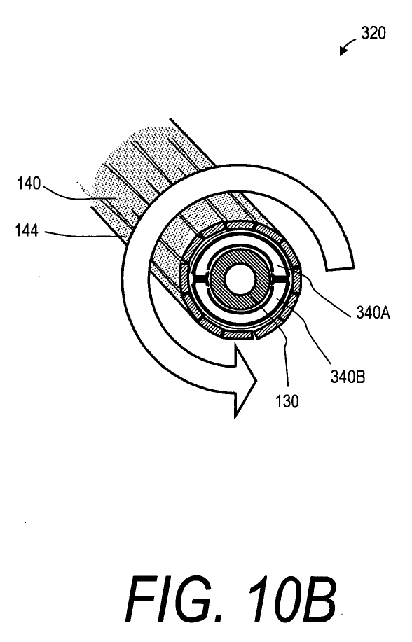

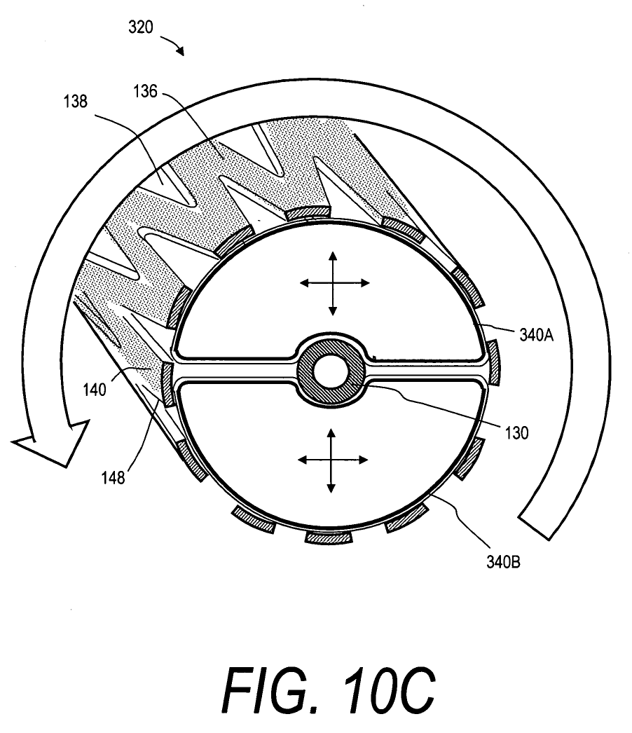

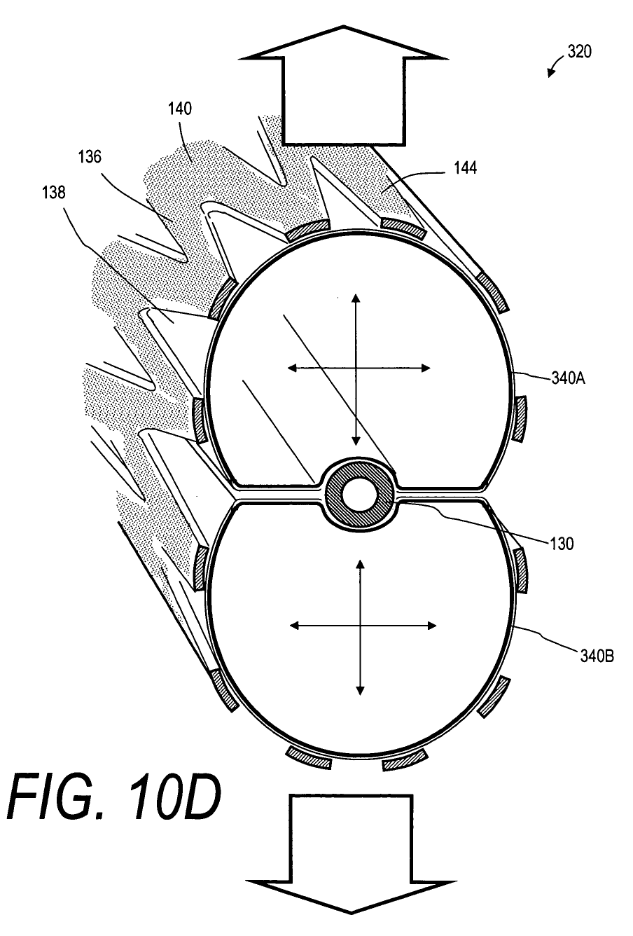

As with the rotatable stent 120 illustrated in FIGS. 4A-4E, in one embodiment the rotatable asymmetric stent 320 as shown in FIGS. 10A-10D can comprise a metal scaffold with struts 136 that circumscribe openings 138. In a collapsed configuration, the stent body 320 preferably has diameter D at a transverse cross-section of between about 3 mm and about 5 mm. In an expanded configuration, the stent 320 preferably has a diameter D′ at a transverse cross-section of between about 10 mm and about 20 mm, as described above with respect to FIGS. 4A-4C.

Of particular interest, as can be seen in FIGS. 7, 8A-8C and 10A, the stent 320 has an outer surface 140 that includes surface features 144 adapted for abrasive removal, grinding or cutting of cancellous bone upon high speed rotation of the spin stent 320 as it is expanded. The surface features 144, in an exemplary embodiment, comprise abrasive surface features such as abrasive particles of diamond, carbide or other materials affixed to the surfaces 140. Diamond particles can be natural monocrystalline diamond, synthetic monocrystalline diamond, polycrystalline diamond or a combination thereof. Diamond particles can have a mean dimension ranging from about 0.25 micron to 100 microns, and more preferably from about 1 micron to 50 microns. The edges 148 of the openings 138 can further be sharpened to function as surface cutting features. As indicated in FIG. 7, a first end of shaft 130 is operatively coupled to the drive motor 125 for rotating the shaft at a speed ranging from about 100 rpm to 50,000 rpm or more, and preferably from about 500 rpm to 10,000 rpm. Under higher speed rotations, the spinning of the stent can apply substantial centrifugal forces as a component of the forces required to move the stent to the expanded condition.

Referring now to FIGS. 10A-10D, an exemplary method for treating a vertebral body is shown. In the illustrated embodiment, the stent 320 comprises a scaffolding structure composed at least primarily of metal having an unexpanded condition and an expanded condition with an asymmetric cross-section, wherein surface cutting features 144 of the stent 320 are used for high speed rotational reduction of cancellous bone. The stent 320 is introduced into the cancellous bone in the unexpanded condition and is preferably contemporaneously rotated and expanded using first and second balloons 340A, 340B to cut, grind or otherwise remove cancellous bone. In another embodiment, the stent 320 can be expanded during intermittent stops in the rotation of the stent 320. Preferably, the stent 320 is in an expanded configuration to support the bone of the vertebral body when rotation of the stent 320 terminates. The stent 320 is further expanded with the first and second balloons 340A, 340B toward an asymmetric configuration to apply vertical forces to augment the height of the vertebra. Following deployment of the rotatable or spin stent 320, the stent 320 preferably supports the bone to prevent its subsidence. The bone can subsequently be infilled with a bone cement or bone graft material, as described above.

FIG. 10A shows the working end 126 of the probe 110 introduced into the cancellous bone 122 of a vertebra, with the stent 320 in the contracted or pre-deployed configuration. FIGS. 10B and 10C illustrate the contemporaneous high speed rotation and initial expansion of the balloons 340A, 340B and stent 320. In one embodiment, the fluid inflow and aspiration sources 135A, 135B can be actuated, as discussed above with regard to FIG. 3, to irrigate and suction the treatment site about the stent surface 140 to remove cut bone debris from the treatment site. Preferably, during said high speed rotation of the stent 320 and the cutting of bone, the stent 320 and dual balloon expansion system causes the stent 320 to maintain a substantially round transverse cross-sectional shape, as shown in FIGS. 10B and 10C. In one embodiment, asymmetric expansion forces from the two balloons are retrained to substantially symmetric forces by the high speed rotation. When the rotation of the cutting stent 320 is ceased, the partially expanded stent 320, as shown in FIG. 10C, preferably maintains the vertebra in a distracted state that substantially prevents any subsidence of vertebral height.

As illustrated in FIG. 10D, the stent 320 can be used to apply asymmetric distraction forces to augment or restore vertebral height. In FIG. 10D, each of the balloons 340A, 340B is expanded further. As a result of said expansion, each of the balloon 340A, 340B tends toward a more round transverse cross section, in turn causing the stent 320 to expand to an asymmetric configuration. Preferably, prior to said further expansion, the balloons 340A, 340B are oriented to distract bone in a desired direction. For example, the balloons 340A, 340B can be oriented vertically, as shown in FIG. 10D. In one embodiment, the balloons 340A, 340B can be oriented in the desired direction using an indicator on the shaft or inner sleeve 130 viewed through port 166 in handle 124 (see FIG. 7). In a preferred embodiment, the handle 124 includes a portion 170 selectively engageable and disengageable from the shaft 130 for manually rotating the balloon-stent assembly 320 to the desired angular orientation.

In the asymmetric expanded configuration, the stent 320 preferably has a greater vertical cross-sectional dimension and a lesser horizontal cross-sectional dimension. The cross-sectional configuration of the stent 320 is preferably non-round, and may be generally oblong, oval, elliptical or partially rectangular.

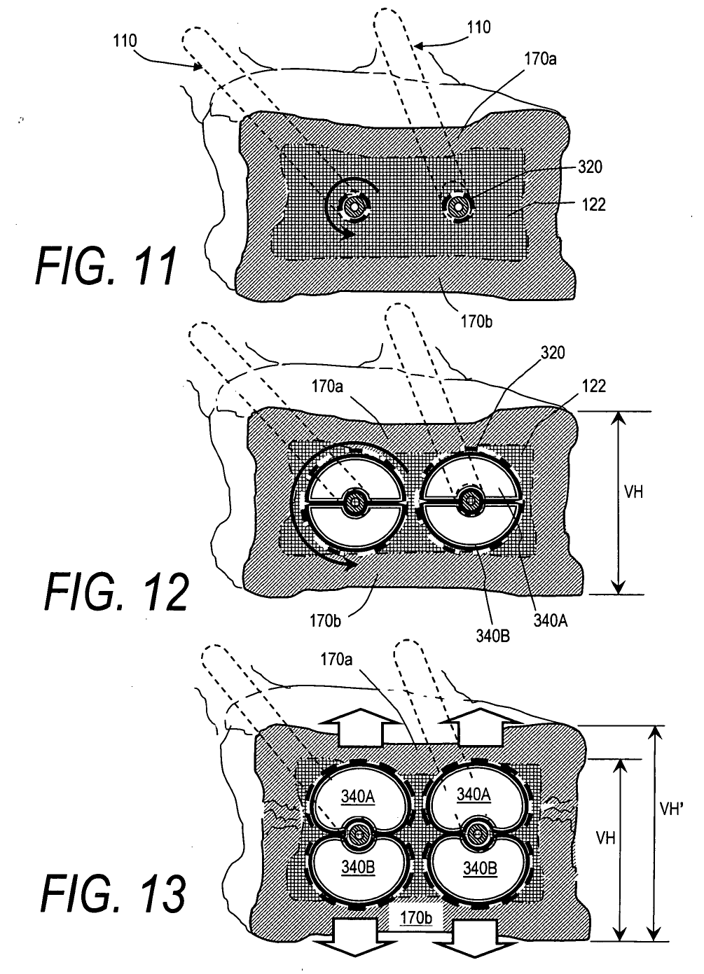

FIGS. 1-13 illustrate similar views of one embodiment of a method for treating a vertebral body, wherein the vertebra has an initial vertebral height VH. The stent deployment system 100 is actuated to rotate the stent 320 in order to cut and remove cancellous bone 116, preferably until the stent 320 engages cortical bone 170a, 170b. As shown in FIG. 9 and discussed above, following the termination of stent rotation and the positioning of the stent 320 in the desired angular orientation, the balloons 340A, 340B are expanded to provide distraction forces to the vertebra and achieve an increased vertebral height to VH′. Following the expansion of the stent 320 to the asymmetric shape of FIG. 13, the stent 320 preferably maintains the vertebra in the augmented height VH′.

In one preferred embodiment, the balloon expansion source 145 is configured to generate a suitable pressure in the balloons 340A, 340B for elevating the vertebral height and, if desired, for fracturing the callus bone about an old fracture. Preferably, the balloon expansion source 145 can apply a pressure, and the balloons 340A, 340B are preferably configured to withstand said pressure, in the range of between about 50 psi and about 500 psi to elevate the vertebral height and/or fracture callous bone.

In a subsequent step (not shown), a fill material M can be inserted into at least one of the balloons 340A, 340B, or into the center 150 of stent 320 with the balloons 340A, 340B collapsed. As discussed above with respect to FIG. 4D, the fill material M can flow through the openings 138 of the stent 320 in a plume to intercalate with cancellous bone 122. In one embodiment, the balloons 340A, 340B are deflated before the fill material M is inserted into the stent 320. In another embodiment, the balloons 340A, 340B are filled with the fill material M. In still another embodiment, fill material M can be introduced into the stent 320 while the balloons 340A, 340B are expanded, wherein the flow of the fill material M will cause the balloons 340A,340B to collapse.

After expansion of the stent 320 such as shown in FIG. 10D or 13, the distal portion 176b of the introducer and inner sleeve can be de-coupled from the proximal portion 176a, such as described with respect to FIG. 3 above.

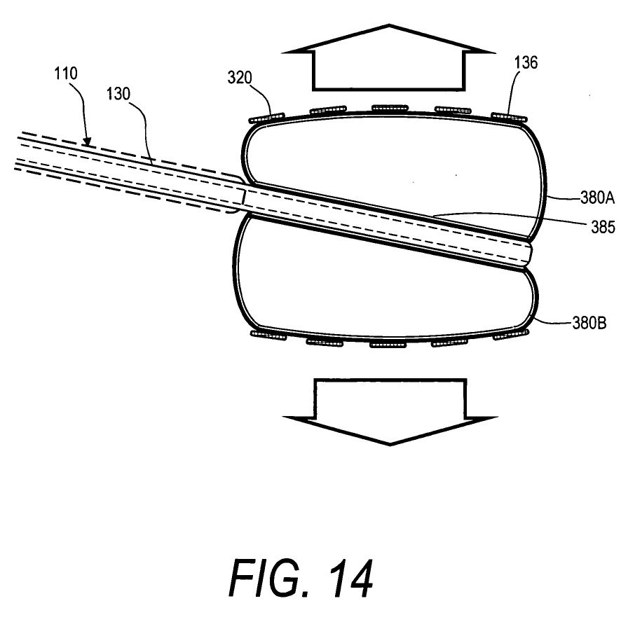

FIG. 14 illustrates another embodiment of stent 320 that is expanded by first and second tapered balloons 380A, 380B. The tapered balloons 380A, 380B preferably generate greater vertical distraction forces for moving the cortical endplates of a vertebra due to the angle of the interface 385 between the balloons 380A, 380B, which generally corresponds to the axis of transpedicular access to the interior of the vertebra.

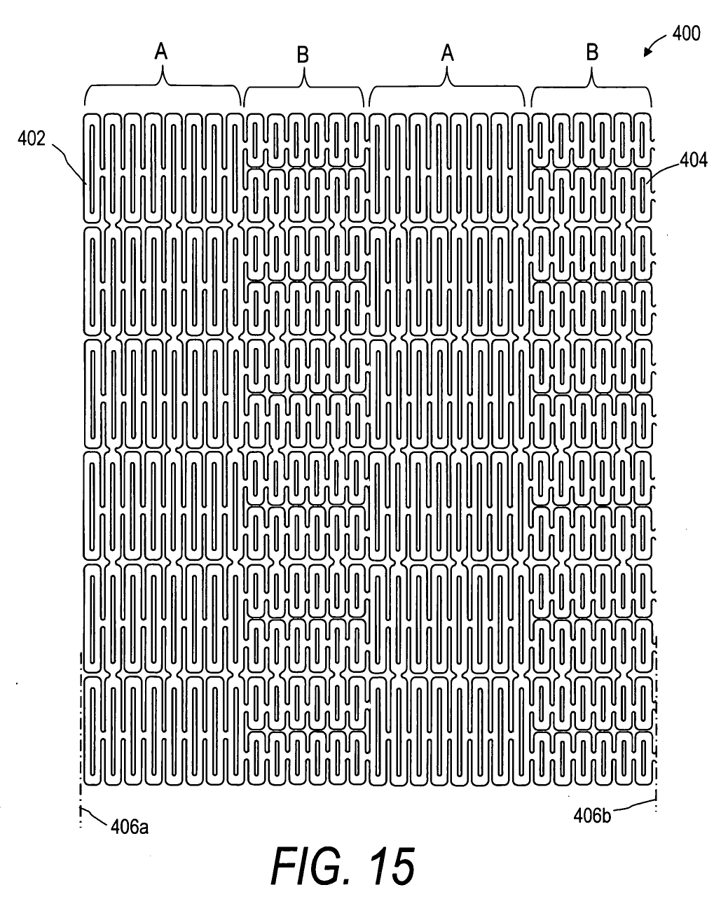

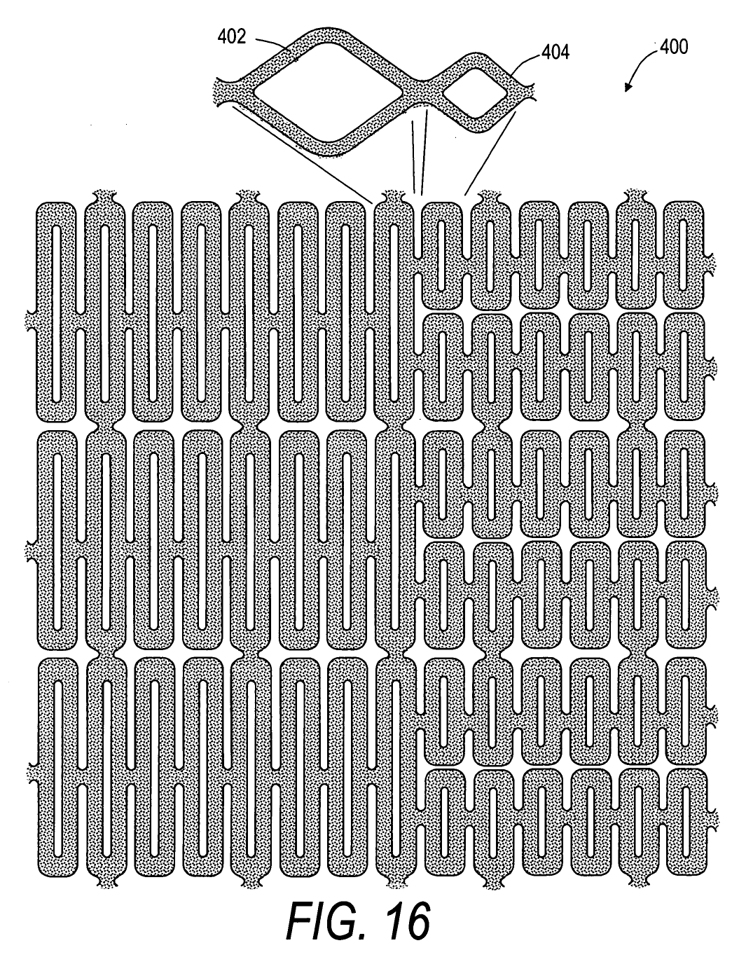

Referring to FIGS. 15 and 16, in one embodiment, the stent 320 is fabricated of a slotted metal 400 having a pattern adapted to expand asymmetrically at least in part due to the differences in expandability of different portions A, B of the stent 320. Differing patterns may be provided along different radial regions around the circumference of the stent. Such differences in expandability can be achieved via, for example, different length struts 402, 404. For example, shorter struts may constrain expandability. In another embodiment, the variability of expandability can also be provided by varying the thickness of the metal struts. FIG. 16 illustrates one example of the approximate maximum expansion of the different length struts 402, 404, which can enable or enhance cross-sectional asymmetry in the balloon expansion of the stent 320. In the illustrated embodiment, the slotted metal can be a perforated metal 200 formed into a cylinder by joining the material along lines 206a, 206b.

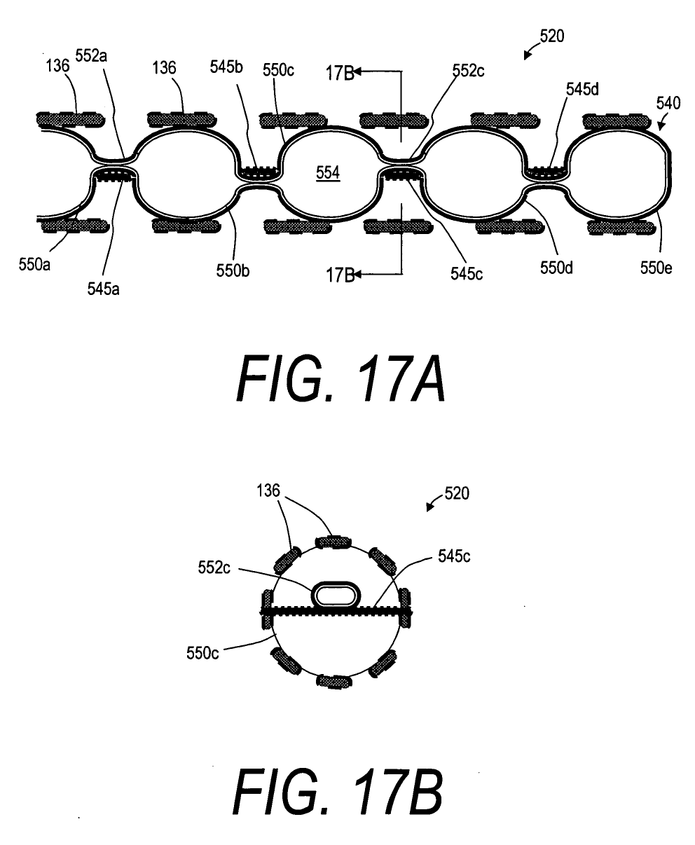

With reference to FIGS. 17A-17B, another embodiment of a rotatable asymmetric stent 520 is shown, having at least one balloon member 540 and at least one interior restraint or structure 545 disposed therein. In the illustrated embodiment, one balloon member 540 is shown. Where components are similar to components in any of the embodiments discussed above, the same numerical identifier is used.

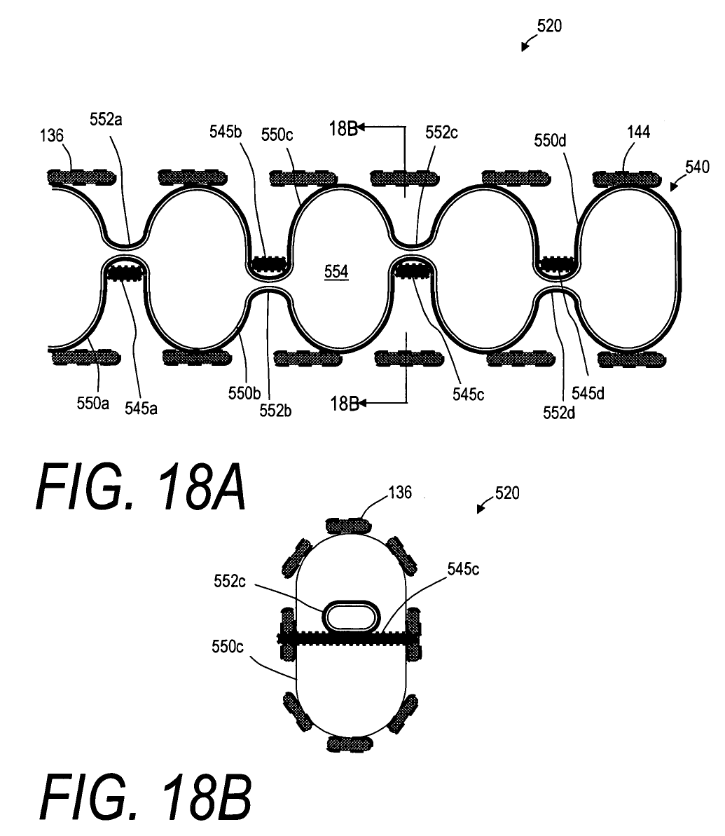

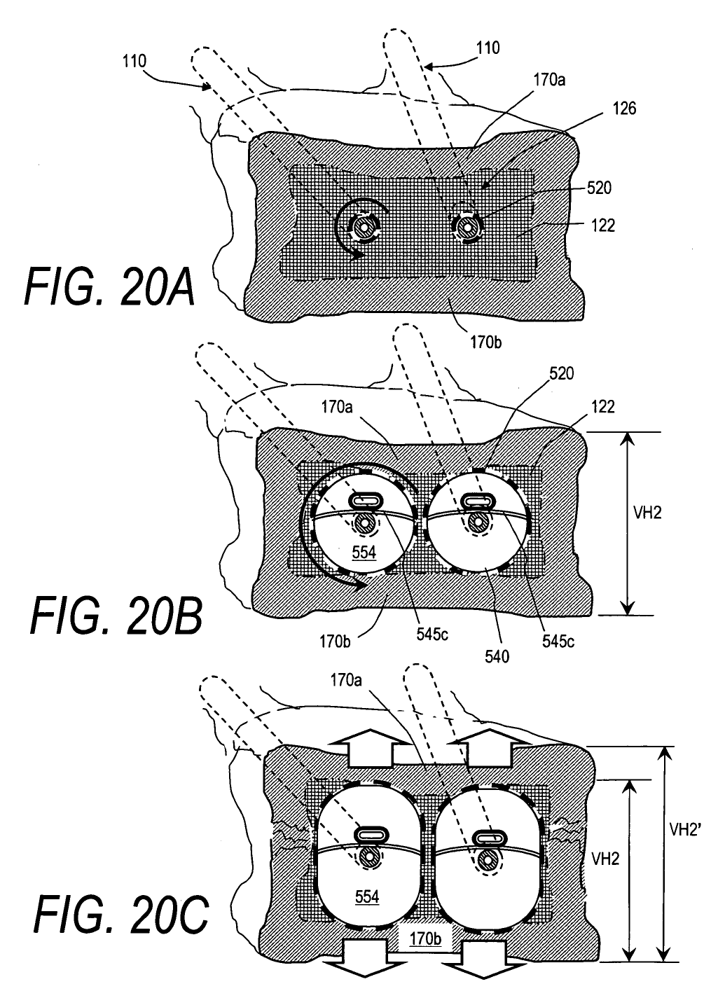

The stent 520 may be deployed using a stent deployment system such as described with respect to FIG. 7 above. Fluid pressure source 145 may be used for expanding the balloon member 540 as will be described in detail below. The system allows for expansion of the stent body 520 in two phases. In a first expansion phase, depicted in a longitudinal and transverse sectional views in FIGS. 17A-17B, the stent 520 is expanded from a pre-deployed non-expanded configuration (such as shown in FIG. 7) to an expanded symmetric configuration (i.e., round in cross-section). In this first expansion phase of FIGS. 17A-17B, the stent 520 is rotated to cut cancellous bone, as also depicted in FIGS. 20A-20B. During the cutting step, the high speed rotation against bone also assists in maintaining the stent in the round cross-section of FIG. 17B. The cutting stent is used to cut cancellous bone and remove debris until the stent's cutting surface engages the cortical bone of the endplates 142a and 142b. In a second stent expansion phase, depicted in a sectional views in FIGS. 18A-18B, the stent 520 is not under rotation for cutting. The stent body 520 then is expanded from the partially expanded configuration of FIG. 17B to an expanded asymmetric configuration as depicted in FIGS. 18A-18B. Before expanding the stent 520 as in FIGS. 18A-18B, the stent and or introducer is rotated by hand to correctly orient the potential asymmetric vertical cross-section to allow application of distraction forces against the cortical bone of the endplates 142a and 142b as shown in FIGS. 20B-20C

Now turning to the schematic views of FIGS. 17A-17B, it can be seen that the stent 520 has an interior restraint 545 preferably configured to restrain the expansion of the stent body 520 in a particular direction when the stent 520 has expanded beyond a certain cross-sectional dimension—thus providing the asymmetric aspect of the stent 520. In the embodiment illustrated in FIGS. 17A-18B, the interior restraint 545 includes a plurality of spaced apart interior restraints or restraining elements 545a-545d. In one embodiment, the number of restraining elements may number from between about 1 and about 10 restraints. In another embodiment, the number of restraining elements can be more than 10. The interior restraints 545a-545d are preferably fabricated of a non-distensible or non-stretchable material that can be folded to allow the collapsed configuration of the stent 520, as seen in FIGS. 3 and 7. In a preferred embodiment, the interior restraints 545a-545d are made of a wire or string-like material. However, in other embodiments the interior restraints 545a, 545b can be made of a mesh, knit, woven, or braided material. The interior restraints can also be made of nickel titanium filaments, polymer filaments or a combination thereof.

With continued reference to FIGS. 17A-17B, in one embodiment the balloon member 540 can be expanded using the expansion source 145, as discussed above with respect to FIG. 7. Preferably, the balloon member 540 is configured to expand the stent 520 through a range of symmetric cross-sectional shapes, illustrated in FIGS. 17A-17B, and a range of asymmetric cross-sectional shapes, as illustrated in FIGS. 18A-18B. In a preferred embodiment, the balloon 540 is configured to extend around or about the spaced apart interior restraints 545a-545d, and comprises cooperating bulb-shaped portions 550a-550d that transition to intermediate necked-down portions 552a-552d. In at least one embodiment, the balloon 540 has an interior chamber 554 that expands the stent 520, as shown in FIGS. 17A-18B, 19A-19B and 20A-20C. In another embodiment, there can be multiple interior chambers 554. The balloon 540 is preferably fabricated of a non-distensible material known in the art, such as PET or urethane. One such balloon suitable for treating a vertebral body according to any of the embodiments disclosed herein is fabricated by Advanced Polymers, Inc., 13 Industrial Way, Salem, N.H. 03079.

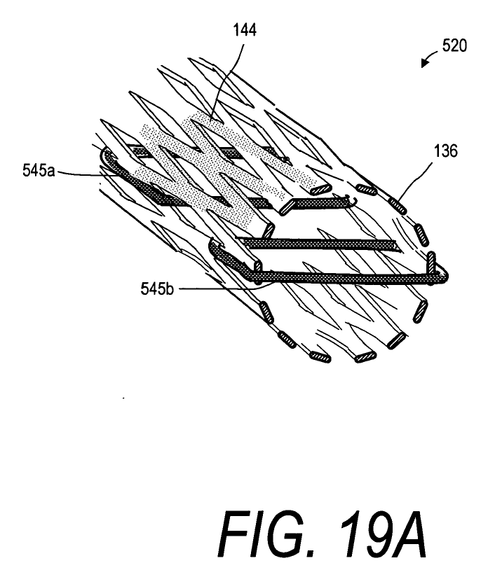

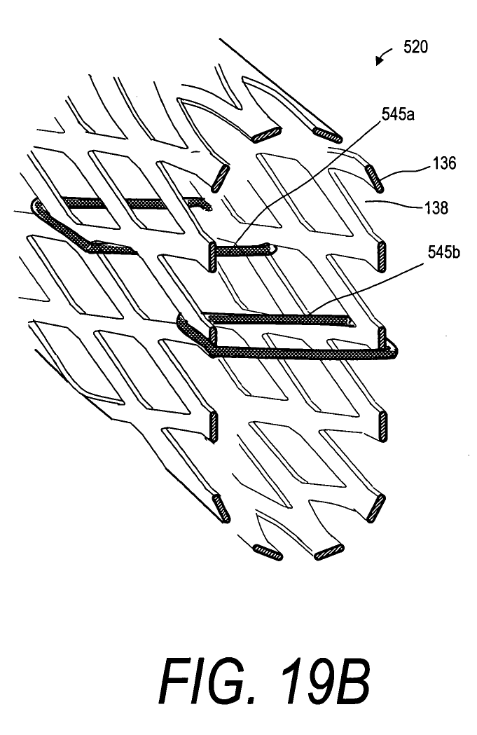

FIGS. 19A and 19B illustrate a sectional perspective view of the stent 520 in a partially expanded symmetric configuration and in a further expanded asymmetric configuration, respectively. The balloon 540 is not shown in FIGS. 19A-19B to provide a better view of one embodiment of the spaced apart interior restraints 545a, 545b. In this embodiment, the interior restraints 545a, 545b are of a flexible but non-distensible filament that extends from one side to another side of the stent body 520. Preferably, the interior restraints 545a, 545b fold or crumple when the stent 520 is collapsed to the pre-deployed configuration shown in FIGS. 3 and 7.

Irrigation and aspiration can optionally be provided through the stent 520 in the manner discussed above in conjunction with FIGS. 3, 7 and 9. In one embodiment, irrigation and aspiration are provided from the fluid inflow and aspiration sources 155A, 155B, respectively, through lumens in the shaft or inner sleeve 130. Preferably, the fluid inflow is introduced at a distal end of the stent 520 assembly, while debris is extracted at the proximal end of the stent assembly 520. However, other suitable irrigation and aspiration configurations can be used.

As with the rotatable stent 120, 320 illustrated in FIGS. 4A-4D, 10A-10D, the rotatable asymmetric spin stent 520 shown in FIGS. 19A-19B can in one embodiment comprise a metal scaffold with struts 136 that circumscribe openings 138. The body of the stent 320 preferably has a structure collapsible to a suitable diameter D akin to a slotted tube shown in FIGS. 3 and 7. Additionally, the abrasive surface features 144 of the stent 520 are configured for the abrasive removal, grinding or cutting of cancellous bone upon high speed rotation of the rotatable or spin stent 520. In one embodiment, the surface features 144 are abrasive particles bonded onto the outer surface 140 of the stent 520. Preferably, the stent 520 is rotated as it is expanded to the symmetric configurations. In another embodiment, the stent. 520 is intermittently expanded during stops in the rotation of the stent 520.

Referring now to FIGS. 20A-20D, an exemplary method for treating a vertebral body is shown. In the illustrated embodiment, the cutting stent 520 has a scaffold-like structure composed at least primarily of metal, as discussed above. The stent 520 preferably has an unexpanded configuration and is capable of expansion to an expanded configuration with an asymmetric cross-section.

The asymmetric stent 520 is preferably introduced into cancellous bone in the unexpanded configuration, as discussed above, and is contemporaneously rotated and expanded in a symmetric cross-sectional configuration to cut, grid or otherwise remove cancellous bone. The stent 520 is preferably in the expanded symmetric configuration that supports the bone of the vertebral body when the rotation of the stent 520 terminates. If necessary, the stent 520 can be further rotated to a desired angular orientation and further expanded to an asymmetric configuration to apply vertical forces to augment the height of the vertebra. Following deployment of the cutting stent 520, the stent 520 preferably supports the bone to prevent its subsidence. The bone can subsequently be infilled with a bone cement, graft material or other suitable fill material.

FIGS. 20A-20C depict one embodiment of a method for treating a vertebral body having an initial vertebral height VH2. The working end 126 of the stent deployment system 100, with the stent 520 in the collapsed or pre-deployed configuration is introduced into cancellous bone 122. The system 100 is activated to cut and remove cancellous bone 122 via high speed rotation of the stent 520, preferably until the cutting stent 520 engages cortical bones 170a, 170b. In FIG. 20B, the contemporaneous rotation and initial expansion of the balloon 540 and stent 520 can be accompanied by fluid inflows and outflows from the fluid and aspiration sources 155A, 155B, respectively, as discussed before. In a preferred embodiment, during the high speed rotation of the stent assembly 520 and the cutting of bone, the balloon expansion system 145 causes the stent 520 to maintain a substantially round cross-sectional shape. When the rotation of the cutting stent 520 is stopped, the partially expanded stent 520 will support the vertebra in the then-existing shape and prevent any subsidence of vertebral height.

FIG. 20C illustrates how the working end 126 and stent 520 can be used to apply asymmetric distraction forces to augment or restore vertebral height. The stent 520 and balloon assembly 540 are preferably oriented to allow asymmetric expansion in the vertical direction. Said orientation can be determined by an indicator on the shaft or inner sleeve 130 viewed through the port 166 in the handle 124, as discussed above with respect to FIG. 7. FIG. 20C thus depicts the termination of stent 520 rotation and the expansion of balloon 540 to achieve an increased vertebral height VH2′. The pressure in the balloon 540 can range from between about 50 psi and about 500 psi to elevate the vertebral height and/or fracture callous bone about an old fracture, if required. Following the expansion of stent 520 to the asymmetric configuration, the stent 520 preferably maintains the vertebra in the augmented height.

Like the embodiment shown in FIGS. 10D and 13 above, in the asymmetric expanded configuration, the stent 520 preferably has a greater vertical cross-sectional dimension and a lesser horizontal cross-sectional dimension. The cross-sectional configuration of the stent 520 is preferably non-round, and may be generally oblong, oval, elliptical or partially rectangular.

A fill material can then be introduced into the balloon 540, as discussed previously. In another embodiment fill material can be introduced into the interior of the expanded stent 520, which will collapse the balloon 540. In yet another embodiment, as discussed above, fill material can be introduced into both the interior of the balloon 540 and about the balloon 540 exterior, where the fill material can flow through the openings 138 of the stent 520 in a plume to intercalate with cancellous bone 122. Depending on the selected diameter D′ of stent 520 in its expanded configuration, the stent 520 can reach the superior and inferior cortical bone (endplate) layers 170a, 170b of the vertebra. In another embodiment, the stent 520 can be somewhat smaller to leave a margin of cancellous bone 122 around the stent 520. The stent 520 may be decoupled from the probe or introducer as described above.

In one embodiment, the system described above can also be used to reinforce osteoporotic vertebrae in a prophylactic manner. Advantageously, the stent 120, 320, 520 can be used to cut cancellous bone and to expand to any suitable dimension, which need not be full expansion.

In any of the above methods, the volume of bone cement used can comprise PMMA, monocalcium phosphate, tricalcium phosphate, calcium carbonate, calcium sulphate and hydroxyapatite, or any combination thereof. Preferably, the bone cement can also carry allograft material, autograft material, or any other infill bone, infill granular material or scaffold material as in known in the art. In at least one of the embodiments discussed above, the volume of bone cement can carry a radiopaque material. Additionally, in at least one of the embodiments discussed above, the volume of bone cement can carry a selected chromophore for cooperating with a light source wavelength in order to accelerate the hardening of the bone cement.

In any bone cement used with any of the embodiments discussed above, there can be infill materials that include polymeric materials configured for timed release of a pharmacological or bioactive agent (e.g., any form of bone morphogenic protein (BMP), an antibiotic, an agent that promotes angiogenesis, etc.). In another example, scaffold elements can be included that are fabricated by e-spinning methods disclosed in co-pending Provisional U.S. Patent Application Ser. No. 60/588,728 filed Jul. 16, 2004 titled Orthopedic Scaffold Constructs, Methods of Use and Methods of Fabrication, the contents of which are incorporated herein in their entirety and should be considered part of this specification.

In any of the embodiments discussed above, the stent deployment system 100 and stent 120, 320, 520 are preferably sterilized for use in the treatment of bone, and particularly vertebral bodies. For example, in one embodiment the stent 120, 320, 520 can be autoclaved. However, the stent deployment system 100 can be sterilized via any suitable mechanism known in the art.

The above description of the invention is intended to be illustrative and not exhaustive. Particular characteristics, features, dimensions and the like that are presented in dependent claims can be combined and fall within the scope of the invention. The invention also encompasses embodiments as if dependent claims were alternatively written in a multiple dependent claim format with reference to other independent claims. Specific characteristics and features of the invention and its method are described in relation to some figures and not in others, and this is for convenience only. While the principles of the invention have been made clear in the exemplary descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

1. A method for treating a vertebral body, comprising:
inserting a stent into the vertebral body in substantial contact with cancellous bone;
rotating the stent to cut cancellous bone from the vertebral body; and
expanding the stent to support the vertebral body.

2. The method of claim 1, wherein the stent is rotated and expanded simultaneously.

3. The method of claim 1, wherein the stent is expanded to a symmetric configuration.

4. The method of claim 1, wherein the stent is expanded to an asymmetric configuration.

5. The method of claim 1, wherein the stent is first expanded to a symmetric configuration, and is then expanded to an asymmetric configuration.

6. The method of claim 1, wherein introducing the stent includes introducing the stent through a pedicle to the vertebral body.

7. The method of claim 1, wherein the stent is introduced into the vertebral body minimally invasively.

8. The method of claim 1, further comprising:
irrigating the vertebral body; and
suctioning cut bone debris from the vertebral body.

9. The method of claim 1, wherein expanding the stent includes expanding the stent into substantial contact with cortical bone endplates of the vertebral body.

10. The method of claim 1, further comprising inserting the stent with an introducer, and detaching the stent from the introducer following expansion.

11. The method of claim 1, wherein the stent is rotated at a speed of between about 100 rpm to about 50,000 rpm.

12. The method of claim 1, further comprising introducing a fill material through the stent and into the vertebral body.

13. The method of claim 1, wherein expanding the stent includes expanding at least one balloon disposed within the stent.

14. The method of claim 1, comprising:
inserting a plurality of stents into the vertebral body in substantial contact with cancellous bone;
rotating the stents to cut cancellous bone from a treatment site; and
expanding the stents to support the vertebral body.

15. A method for treating a vertebral body, comprising:
inserting a stent into the vertebral body, the stent having a collapsed configuration and an expanded configuration;
cutting cancellous bone with the stent;
expanding the stent within the vertebral body; and
releasing the stent such that the stent remains in place to support the vertebral body.

16. The method of claim 15, comprising rotating the stent to cut the cancellous bone.

17. The method of claim 15, wherein the stent is expanded simultaneously with cutting the cancellous bone.

18. The method of claim 15, wherein the stent is made of metal.

19. The method of claim 15, further comprising injecting bone cement into the stent after expansion.

20. The method of claim 19, further comprising directing bone cement through openings in the stent and outside the stent to support the vertebral body.

21. The method of claim 15, wherein the stent is inserted on the end of an introducer.

22. The method of claim 21, wherein the stent is carried on an inner shaft extending through an elongated shaft of the introducer.

23. The method of claim 22, wherein the stent is expanded by drawing proximal and distal ends of the stent closer together.

24. The method of claim 22, wherein the inner shaft is rotatable to cut the cancellous bone with the stent.

25. The method of claim 21, wherein releasing the stent comprising releasing the introducer from the stent.

26. The method of claim 15, further comprising delivering a balloon into the expanded stent after injecting bone cement into the stent, and expanding the balloon against hardened bone cement.

27. The method of claim 15, wherein the stent is expanded with at least one balloon.

28. A method for treating a vertebral body, comprising:
inserting a stent into the vertebral body;
expanding the stent asymmetrically such that the stent applies a greater expansion force along an axis extending generally between two cortical end plates of the vertebral body than within a plane generally parallel to the two cortical end plates; and
releasing the stent such that the stent remains in place to support the vertebral body.

29. The method of claim 28, further comprising using the stent to cut cancellous bone from within the vertebral body.

30. The method of claim 29, further comprising using at least one of irrigation and aspiration to remove cut bone material.

31. The method of claim 28, further comprising expanding the stent symmetrically before expanding the stent asymmetrically.

32. The method of claim 28, wherein the stent is expanded with at least one balloon.

33. The method of claim 28, wherein the stent is expanded with two balloons.

34. The method of claim 32, wherein a restraint is provided around at least a portion of the balloon to cause the stent to expand asymmetrically.

35. The method of claim 28, wherein the stent is rotated to align said stent before asymmetrical expansion.

36. A method for treating a bone, comprising:
introducing an expandable stent having surface abrasives into an interior of the bone;
spinning the stent to cut the bone; and
expanding the stent;
wherein the stent after spinning provides bone support to prevent subsidence.

37. The method of claim 36, wherein spinning and expanding the stent occur simultaneously.

38. The method of claim 36, further comprising irrigating and aspirating cut bone debris.

39. The method of claim 36, wherein expanding the stent is accomplished by forces applied by at least one of a mechanical stent-expansion mechanism, a balloon stent-expansion mechanism, the release of energy stored in a shape memory stent body, and centrifugal force.

40. The method of claim 36, further comprising filling the interior of the bone with at least one of a bone cement, bone allograft or bone autograft.

41. The method of claim 36, comprising introducing the expandable stent into a vertebral body.

42-117. (canceled)
